(19) United States
(12) Patent Application Publication
(10) Pub. No.: US 2012/0107317 A1
(43) Pub. Date: May 3, 2012

(54) USE OF CYTOPLASMIC C-MYC FOR REGULATING IMMUNE RESPONSES

(75) Inventors: ALLAN SIK YIN LAU, Hong Kong (CN); Howard Chi Ho Yim, Hong Kong (CN); Chun Bong Li, Hong Kong (CN); John Chi Him Pong, Hong Kong (CN)

(73) Assignee: The University of Hong Kong, Hong Kong (CN)

(21) Appl. No.: 13/282,084

(22) Filed: Oct. 26, 2011

Related U.S. Application Data
(60) Provisional application No. 61/407,276, filed on Oct. 27, 2010.

(51) Int. Cl. A61K 39/395; G01N 33/566; G01N 33/573; A61K 31/713; A61K 31/424; A61K 31/5383; A61K 31/196; A61K 31/444; A61K 38/17; A61P 35/00; A61K 31/7088; A61P 29/00; A61P 37/06; A61P 37/08; A61P 9/00; A61P 31/18; A61P 31/04; A61P 31/10; A61P 31/12; A61P 31/20; A61P 31/22; A61P 37/04; C12Q 1/68

(52) U.S. Cl. 424/138.1; 435/6.12; 435/7.1; 435/7.92; 435/7.4; 435/6.1; 514/44 A; 514/375; 514/229.5; 514/563; 514/333; 514/1.1; 514/13.3; 514/19.3; 424/278.1

(57) ABSTRACT

The subject invention provides novel uses of cytoplasmic c-Myc for modulation of innate immune responses. The invention is based, at least in part, on the surprising discovery that cytoplasmic c-Myc, instead of nuclear c-Myc, modulates pro-inflammatory immune responses via its role as a positive feedback regulator. Specifically, the subject invention provides methods for treatment or amelioration of inflammatory diseases and/or immune disorders via inhibition c-Myc expression or its activity. Also provided are methods for the development of therapeutic agents for treating infection, inflammation, immune diseases and autoimmune diseases.

### CROSS-REFERENCE TO RELATED APPLICATION

The subject application claims the benefit of U.S. Provisional Application Ser. No. 61/407,276, filed Oct. 27, 2010, which is incorporated herein by reference in its entirety.

### BACKGROUND OF THE INVENTION

Mycobacteria such as Mycobacterium tuberculosis (M. tuberculosis) and Mycobacterium avium (M. avium ) are common opportunistic pathogens that cause disseminated infections, particularly in individuals with compromised immune system function such as in patients in late stage of AIDS (Karakousis et al., 2004). Tuberculosis alone causes 1.8 million deaths worldwide in 2008 (World Health Organization, 2009).

Upon infection, hosts mount inflammatory reactions to control the pathological conditions. The invading pathogens are first recognized by specific host receptors including toll-like receptor 2 (TLR2), TLR4, TLR9, dectin-1 and nucleotide-binding oligomerization domain 2 (NOD2) (Ferwerda et al., 2005; Jang et al., 2004; Underhill et al., 1999; Yadav and Schorey, 2006), leading to the activation of signaling cascades and the expression of cytokines for innate and adaptive immunity.

For example, the activated TLR2 or TLR4 recruits MyD88, IRAK1 and IRAK4. IRAK4 phosphorylates IRAK1, which in turn binds to TRAF6 (Moynagh, 2009; O'Neill and Bowie, 2007). Upon binding, TRAF6 then recruits other signaling molecules, including TAB1, TAB2, TAB3 and TAK1, leading to the activation of IKK complex (Moynagh, 2009; O'Neill and Bowie, 2007). The IKK complex induces the phosphorylation of IκBα, which is then degraded to release NFκB p50/p65 for nuclear translocation to induce the expression of cytokines including TNF-α and IL-6 (Ghosh and Hayden, 2008; Moynagh, 2009; O'Neill and Bowie, 2007). The IKK complex also phosphorylates p105, resulting in the activation of Tpl2, which activates ERK1/2—a mitogen-activated protein kinase (MAPK) (Banerjee et al., 2006; Cho et al., 2005; Cho and Tsichlis, 2005; Moynagh, 2009). Additionally, TAK1 activates MAPKs, including p38 kinase and JNK, via the activation of MEKK3 (Huang et al., 2004; Moynagh, 2009). The present inventors have also delineated that mycobacteria activate ERK1/2 and p38 kinase via dsRNA-activated protein kinase PKR and MAPK-phosphatase-1 (MKP1) (Cheung et al., 2005; Cheung et al., 2009). Activation of these kinases triggers transcription factors including CREB and AP-1 to stimulate the expression of cytokines such as IL-10 and TNF-α (Hu et al., 2006; Kang et al., 2007; Moynagh, 2009).

Human immunodeficiency virus (HIV) dysregulates cytokine responses and suppresses host immune function (Kasper et al., 2005; McMichael et al., 2001; Noursadeghi et al., 2006). HIV proteins that contribute to the subversion of host immunity include Vpr and Tat. Vpr has anti-apoptotic activities (Fukumori et al., 1998) and Tat dysregulates cellular events (Strebel et al., 2003; Li et al., 2005). Specifically, HIV-1 Tat dysregulates host IFN-γ responses, evades TNF-α-induced cytotoxicity (Strebel et al., 2003), and upregulates IL-10, a potent anti-inflammatory cytokine that suppresses T cell function (Li et al., 2005; Mege et al., 2006). For instance, Tat alters the activities of various kinases including PKR (Li et al., 2005). In addition, Tat stimulates SOCS2, which suppresses Stat1 phosphorylation (Li et al., 2005; Cheng et al., 2009). Tat also suppresses the endotoxin-TLR4 pathway, resulting in reduced expression of IFN-β (Yim et al., 2009). In addition, HIV Tat reduces MHC class II antigen expression (Cheng et al., 2009) and interrupts the regulation of autophagy by IFN-γ. Tat also induces the over-expression of IL-10 via the activation of PKR, MAPKs and NF-IL6 (Li et al., 2005; Mege et al., 2006). However, the precise mechanisms through which HIV Tat protein dysregulates host cytokine (such as TNF-α and IL-6) responses have not been fully elucidated.

c-Myc (Myc), which is reported to play a causal role in tumorigenesis, was first identified as the human homologue of v-Myc, an oncogenic protein of avian myelocytomatosis virus strain (Vennstrom et al., 1982). The level of c-Myc protein is regulated by Fbw7 via the ubiquitin-proteasome system (Garrison and Rossi, 2010; Reavie et al., 2010).

c-Myc has been shown to act as a transcription factor to regulate the expression of a broad range of genes for controlling various cellular processes, including cell cycle, cell growth and differentiation, metabolism, protein synthesis, cell adhesion and migration, angiogenesis, chromosomal instability, stem cell renewal, and apoptosis (Meyer and Penn, 2008). c-Myc possesses a N-terminal transactivation domain and a C-terminal basic helix-loop-helix (HLH) leucine zipper (LZ) domain for binding to a CACGTG E-box DNA sequence with Max as heterodimers (Meyer and Penn, 2008). Upon binding to c-Myc, Max becomes associated with various Mxd family members to regulate the Myc-dependent gene transcription (Meyer and Penn, 2008). c-Myc also binds directly to various signaling molecules to modulate their activities. For example, c-Myc binds to transactivation/transformation-associated protein (TRRAP), and thereby recruits histone acetylation complexes to chromatin (McMahon et al., 1998; McMahon et al., 2000). c-Myc also binds with Sp1 and MIZ1 to repress gene expression (Meyer and Penn, 2008).

In addition, although c-Myc knock-out mice are embryonically lethal (Davis et al., 1993), conditional c-Myc knockout mice have been developed to demonstrate that c-Myc plays a role in regulating cell number and body size, regulating percentage of activated T cells entering into the cell cycle, and controlling proliferation of B cells upon stimulation of anti-CD40 and IL-4 (de Alboran et al., 2001; Trumpp et al., 2001). In 2007, c-Myc was found to be one of the four genes for the reprogramming of fibroblasts into induced pluripotent stem (iPS) cells (Okita et al., 2007; Wernig et al., 2007). Although c-Myc is not required for iPS cell generation (Nakagawa et al., 2008), it has recently been found as crucial for controlling the differentiation and self-renewal of hematopoietic stem cells (Garrison and Rossi, 2010; Reavie et al., 2010; Wilson et al., 2004).

Despite its role in transcription regulation, tumorigenesis and hematopoiesis, c-Myc has not previously been reported to play any role in regulating innate immune responses. Specifically, it remains unknown whether c-Myc plays any role in controlling pathogenic infection and/or the development of immune and/or autoimmune diseases. As will be clear from the disclosure that follows, these and other benefits are provided by the subject invention.

### BRIEF SUMMARY OF THE INVENTION

The subject invention provides methods for modulation of pro-inflammatory immune responses associated with cytoplasmic c-Myc activity. It is based, at least in part, on the surprising discovery that cytoplasmic c-Myc, instead of nuclear c-Myc, modulates pro-inflammatory immune responses via its role as a positive feedback regulator.

In an embodiment, the subject invention provides methods for treatment or amelioration of inflammatory diseases and/or immune disorders, particularly diseases or disorders associated with cytoplasmic c-Myc activity. The method comprises administering, to a subject in need of such treatment, an effective amount of an agent or a composition that inhibits or reduces c-Myc transcription/expression, c-Myc protein level in the cytoplasm, and/or its activity in regulating pro-inflammatory pathways.

In another embodiment, the subject invention also provides methods for screening for therapeutic agents for treatment or amelioration of inflammation or immune diseases or disorders, by selecting agents that reduce the level and activity of c-Myc in the cytoplasm and/or inhibit or reduce the induction of pro-inflammatory responses by c-Myc. In a specific embodiment, the subject invention provides methods for screening for agents that increase the level or activity of c-Myc antagonists for reducing inflammation.

The subject invention can be used to treat or ameliorate, or to develop therapeutics in the treatment or amelioration of, inflammatory symptoms in any disease, condition or disorder where reduction of c-Myc level in the cytoplasm and/or inhibition or suppression of cytoplasmic c-Myc-induced pro-inflammatory responses is beneficial.

In certain embodiments, the subject can be used to treat or ameliorate, or to develop therapeutics in the treatment or amelioration of, diseases or conditions including, but not limited to, infection, inflammation, allergenic reactions, diseases associated with cell proliferation, neoangiogenesis, malignancy, and cardiovascular diseases.

In preferred embodiments, the subject invention can be used to treat or ameliorate, or to develop therapeutics in the treatment or amelioration of, multiple sclerosis, ulcerative colitis, rheumatoid arthritis and systemic lupus.

In a preferred embodiment, the subject invention can be used to treat or ameliorate, or to develop therapeutics in the treatment or amelioration of, mycobateria infection. In preferred embodiments, the subject invention can be used to treat or ameliorate, or to develop therapeutics in the treatment or amelioration of, HIV infection, opportunistic infection concurrent with HIV infection, and AIDS-associated disorders.

In addition, the subject invention also provides methods for screening for therapeutic agents for enhancing host immune responses for controlling pathogenic infection and/or for restoring or improving compromised immune system function. The method selects agents that increase the level of c-Myc in the cytoplasm and/or enhance the induction of pro-inflammatory responses by cytoplasmic c-Myc.

In a specific, exemplifying embodiment, the subject invention provides methods for developing therapeutic agents for the treatment or amelioration of mycobateria infection (such as tuberculosis), especially acute stage of mycobacteria infection (such as tuberculosis).

### BRIEF DESCRIPTION OF THE DRAWINGS

FIG. 1 shows that mycobacteria induce c-Myc expression. (A) PBMac were treated with mock or Bacille Calmette-Guérin (BCG) (multiplicity of infection [M.O.I.]=1) for the indicated time. (B) PBMac were treated with mock or M. avium (M.O.I.=20) for the indicated time. (C) PBMac were treated with mock or M. chelonae (M.O.I.=10) or M. kansasii (M.O.I.=1) for the indicated time. (D) PBMac were treated with mock or BCG with indicated M.O.I. for 24 hours. (A-D) Cellular proteins were extracted for Western blot analyses to determine the levels of c-Myc and Actin. The data represent results using PBMac cells isolated from 3 independent blood donors. The value under each lane represents the level of Myc relative to Actin. (E) shows fold induction of Myc mRNA by mycobacteria. PBMac were treated with mock or BCG (M.O.I.=1) for the indicated time. Total RNA was extracted for analyzing the mRNA expression levels by quantitative RT-PCR. The data represent mean+SD from 3 independent blood donors. ‘*’ denotes p<0.05. ‘hr’represents hours.

FIG. 2 shows modulation of mycobacteria-induced c-Myc expression by signaling molecules or kinases. PBMac were pretreated with DMSO, 10 μM inhibitors antagonizing ERK1/2 (PD), JNK (SP) or p38 MAPK (SB), or 15 μg/ml inhibitor against NFκB (CAPE) for 1 hour, followed by treatment with mock or BCG (M.O.I.=1) for 3 hours. Total RNA was extracted for analyzing the mRNA expression levels by quantitative RT-PCR. The data represent mean+SD from 3 independent blood donors. ‘*’ denotes p<0.05. ‘hr’ represents hours.

FIG. 3 shows that c-Myc siRNA decreases the mycobacteria-induced c-Myc protein expression. PBMac were transfected with control or c-Myc siRNA for 48 hours, followed by BCG (M.O.I.=1) treatment for the indicated time. Proteins levels were analyzed by Western blot. The value under each lane represents the protein level of c-Myc relative to actin.

FIG. 4 shows that c-Myc selectively mediates the induction of cytokines by mycobacteria. (A-E) PBMac were transfected with control or c-Myc siRNA for 48 hours, followed by treatment with mock, BCG (M.O.I.=1) or M. avium (M.O.I.=20) for 24 hours. Supernatants were harvested for determining the cytokine levels by ELISA. The data represent mean+SD from 3 (A), 4 (B), 5 (C), 3 (D) and 3 (E) independent blood donors. ‘*’ denotes p<0.05.

FIG. 5 shows that c-Myc selectively enhances mycobacteria-induced transcriptional activation of cytokines (A-C) PBMac were transfected as described in FIG. 4, followed by treatment with mock or BCG (M.O.I.=1) for the indicated time. Total RNA was extracted for assaying the mRNA levels of cytokines by quantitative RT-PCR. The data represent mean+SD from 7 (A), 3 (B) and 5 (C) independent blood donors. ‘*’ denotes p<0.05.

FIG. 6 shows that c-Myc does not affect the survival of mycobacteria. PBMac were transfected with control or c-Myc siRNA for 48 hours, followed by M. avium (M.O.I.=20) treatment for 24 hours. Cells were washed extensively and lysed. The intracellular number of M. avium in the lysates was determined and expressed as 10⁶ CFU per million cells. The data represent mean+SD from 3 independent blood donors.

FIG. 7 shows that c-Myc does not affect the viability of PBMac cells. PBMac were transfected with control or c-Myc siRNA for 48 hours, followed by BCG (M.O.I.=1) treatment for 24 hours. The cell viability was examined by the MTT assay. The data represent mean+SD from 3 independent blood donors.

FIG. 8 shows that c-Myc augments the mycobacteria-induced activation of IRAK1 and MAPK pathways. (A-C) PBMac were transfected with control or c-Myc siRNA as described in FIG. 4, followed by BCG (M.O.I.=1) treatment for the indicated time. Cytoplasmic proteins were analysed by Western blot. p-EKR1/2, p-p38 MAPK and p-Akt denote the phosphorylated forms of ERK1/2, p38 MAPK and Akt, respectively. The value under each lane represents the protein level of IRAK1, p-ERK1/2, p-p38 kinase or p-Akt relative to Actin, ERK1/2, p38 kinase or Akt, respectively. The data represent results from cells isolated from 3 independent blood donors. ‘hr’ and ‘M’ denote hour and mock, respectively.

FIG. 9 showed that c-Myc localizes in the cytoplasm of PBMac. (A) PBMac were treated with mock or BCG (M.O.I.=1) for 5 hours. Cells were fixed and co-labeled with primary antibodies against c-Myc (panels a, e, i and m) and α-tubulin (panels b and f) or NFκB p65 (panels j and n), followed by treatment with corresponding secondary antibodies. Cells were then labeled with DAPI (panels c, g, k and o). Images of cells were analyzed by confocal microscopy. Panels d, h, l and p are merged images of panels a-c, panels e-g, panels i-k, and panels m-o, respectively. The merged two-color image of a and b, e and f, i and j, and m and n were analyzed by ImageJ software (National Institute of Health, USA) with Pearson-Spearman correlation colocalization plug-in. (B) PBMac were treated with mock or BCG (M.O.I.=1) for 5 hours. Cytoplasmic and nuclear proteins were analyzed by Western blot. (A and B) The data represent results from cells isolated from 3 independent blood donors.

FIG. 10 shows that c-Myc suppresses mycobacteria-induction of IκBα protein, and shows that c-Myc binds to IκBα. (A) PBMac were transfected with control or c-Myc siRNA as described in FIG. 4, followed by BCG (M.O.I.=1) treatment for the indicated time. Cytoplasmic proteins were analyzed by Western blot. (B and C) PBMacs were treated with BCG (M.O.I.=1) for the indicated time (B) and for 5 hours (C). Proteins were extracted for immunoprecipitation of c-Myc and/or IκBα by specific antibodies. The immunoprecipitates and the cell lysates were then analyzed by Western blot. (A-C) The data represent results from cells isolated from 3 independent blood donors. ‘#’ denotes the non-specific bands. (A) The value under each lane represents the protein level of IRAK1 or IκBα relative to Actin. ‘hr’ and ‘M’ denote hour and mock, respectively.

FIG. 11 shows that c-Myc enhances mycobacteria-induced NFκB nuclear translocation. Cells were fixed and stained with NFκB p65 antibodies and DAPI. The cells were then analyzed by Cellomics ArrayScan HCS VTI Reader. (A) Representative images from 10 fields per well were shown. (B) The nuclear localization of NFκB p65 was quantified and expressed as nuclear/cytoplasmic intensity ratio. The data represent results from 3 independent blood donors. ‘*’ denotes p<0.05.

FIG. 12 shows the effect of c-Myc on TNF-α-induced NF-kB p65 nuclear translocation. (A) Primary human blood macrophages were transfected with siRNA specific to c-Myc for 48 hours, followed by treatment with or without TNF-α (5ng/m1) for 1 hour. The cells were fixed and stained with DAPI (blue) for nuclei and NF-kB p65 visualization (green). (B) The ratio of NF-kB translocation without siRNA to c-Myc was set at 100.

FIG. 13 shows that HIV-1 Tat induces c-Myc expression in primary human blood macrophages. (A) PBMac were treated with or without Tat (100 ng/ml) for indicated time points (2, 4 or 6 hours). Total RNA was extracted for analyzing c-Myc mRNA expression levels by quantitative RT-PCR. (B) PBMac were treated with HIV-1 Tat (20 or 100 ng/ml) for 24 hours. Cellular proteins were extracted for Western blot analyses to determine the protein levels of c-Myc and actin. The level of actin was measured as a control to indicate the amount of proteins loaded. The data represent results from at least 2 independent donors.

FIG. 14 shows that c-Myc mediates the induction of cytokines by HIV-1 Tat. (A-C) PBMac were transfected with control or c-Myc siRNA for 48 hours, followed by treatment with HIV-1 Tat (100 or 200 ng/ml) for 24 hours. Supernatants were harvested for determining the levels of TNF-α (A), IL-6 (B) and IL-1β (C) by ELISA. The data represent mean±SD from independent blood donors.

### BRIEF DESCRIPTION OF THE SEQUENCES

SEQ ID NO: 1 is a nucleic acid sequence that is useful for the subject invention.

SEQ ID NO: 2 is a nucleic acid sequence that is useful for the subject invention.

SEQ ID NO: 3 is a nucleic acid sequence that is useful for the subject invention.

SEQ ID NO: 4 is a nucleic acid sequence that is useful for the subject invention.

SEQ ID NO: 5 is a nucleic acid sequence that is useful for the subject invention.

SEQ ID NO: 6 is a nucleic acid sequence that is useful for the subject invention.

SEQ ID NO: 7 is a nucleic acid sequence that is useful for the subject invention.

SEQ ID NO: 8 is a nucleic acid sequence that is useful for the subject invention.

SEQ ID NO: 9 is a nucleic acid sequence that is useful for the subject invention.

SEQ ID NO: 10 is a nucleic acid sequence that is useful for the subject invention.

SEQ ID NO: 11 is a nucleic acid sequence that is useful for the subject invention.

SEQ ID NO: 12 is a nucleic acid sequence that is useful for the subject invention.

SEQ ID NO: 13 is a nucleic acid sequence that is useful for the subject invention.

SEQ ID NO: 14 is a nucleic acid sequence that is useful for the subject invention.

SEQ ID NO:15 is an amino acid sequence of human c-Myc protein.

### DETAILED DISCLOSURE OF THE INVENTION

The subject invention provides methods for modulation of pro-inflammatory immune responses associated with cytoplasmic c-Myc activity. In an embodiment, the subject invention provides methods for treatment or amelioration of inflammatory diseases and/or immune disorders, particularly diseases or disorders associated with cytoplasmic c-Myc. The method comprises administering, to a subject in need of such treatment, an effective amount of an agent or a composition that inhibits or reduces c-Myc transcription/expression, c-Myc protein level in the cytoplasm, and/or its activity in regulating pro-inflammatory pathways. In an embodiment, the therapeutic agent or composition of the subject invention increases the level or activity of c-Myc antagonists including, but not limited to, MAD1, MAX, MNT, MXD 1-4 (formerly MAD1, MXI1, MAD3 and MAD4) and MGA.

The subject invention also provides methods for screening for therapeutic agents for treatment or amelioration of inflammation or immune diseases or disorders, by selecting agents that reduce the level of c-Myc and/or inhibit or reduce the induction of pro-inflammatory responses by c-Myc.

The subject invention can be used to treat or ameliorate, or to develop therapeutics in the treatment or amelioration of, inflammatory symptoms in any disease, condition or disorder, where reduction of cytoplasmic c-Myc and/or inhibition or suppression of cytoplasmic c-Myc-induced pro-inflammatory responses is beneficial. Alternatively, the subject invention provides methods for screening for agents that increase the level or activity of c-Myc antagonists for reducing inflammation.

In addition, the subject invention also provides methods for screening for therapeutic agents for enhancing host immune responses, for controlling pathogenic infection, and/or for restoring or improving compromised immune system function. The method selects agents that increase the level of c-Myc and/or enhance the induction of pro-inflammatory responses by c-Myc.

In a specific embodiment, the subject invention provides methods for developing therapeutic agents for the treatment or amelioration of mycobateria infection (such as tuberculosis), especially acute stage of mycobacteria infection (such as tuberculosis).

The subject invention is based, at least in part, on the surprising discovery that cytoplasmic c-Myc, instead of nuclear c-Myc, modulates pro-inflammatory immune responses via its role as a positive feedback regulator. c-Myc expression is upregulated during infection via the activation of ERK1/2 and JNK. The increase in levels of c-Myc augments the expression of pro-inflammatory cytokines such as TNF-α and IL-6. It is also discovered that cytoplasmic c-Myc enhances IRAK1 degradation, leading to the phosphorylation and activation of ERK1/2 and p38 MAPK. It also binds specifically to IκBα and promotes IκBα degradation, thereby augmenting the induction of pro-inflammatory cytokines Cytoplasmic c-Myc also enhances the nuclear translocation of NFκB p65. It is also discovered that cytoplasmic c-Mycis not involved in anti-inflammatory pathways. Specifically, cytoplasmic c-Myc does not affect the levels of IL-10, an anti-inflammatory cytokine that is also upregulated during infection. In addition, cytoplasmic c-Myc does not regulate the phosphorylation and activation of Akt. In addition, cytoplasmic c-Myc does not bind to IRAK1, MyD88 or ERK1/2.

Specifically, it is discovered that c-Myc expression was induced during infection. The subject invention demonstrated that species of mycobacteria with different pathogenicity, such as M. bovis (BCG), M. avium, M. kansasii and M. chelonae, activated c-Myc transcription and protein expression in a time- and dose-dependent manner. The induction of c-Myc expression during infection was also evidenced by uncharacterized microarray data obtained from cells infected with M. avium (Blumenthal et al., 2005; Greenwell-Wild et al., 2002). Infection by Helicobacter pylori (H. pylori) and Salmonella typhimurium (S. typhimurium) have also been reported to trigger c-Myc protein expression in mouse macrophages (Cheng et al., 2005; Seong et al., 2009). However, the precise mechanisms through which c-Myc expression is induced during pathogenic infection and the role of c-Myc expression in innate immunity have not previously been elucidated. Additionally, it was unknown whether c-Myc is involved in inflammation and other biological activities.

It is now discovered that the activation of ERK1/2 and JNK1/2 during infection (such as mycobacteria infection) induces c-Myc transcription. c-Myc transcription is regulated not only by its own promoter, but also by NHE III elements in upstream distal positions and other elements far upstream of the c-Myc promoter (Brooks and Hurley, 2009). It is postulated that the activation of ERK1/2 and JNK1/2 during infection (such as mycobacteria infection) stimulates the binding of various transcription factors, including SP1 and E2F, to these elements, thereby activating c-Myc transcription.

Furthermore, it is now discovered although the increase in c-Myc mRNA levels lasted for a very short time, the increase in c-Myc protein levels lasted up to 24 hours during infection. The increase in c-Myc protein levels augments the induction of expression of various cytokines during infection.

It is also postulated that the stability of c-Myc protein is prolonged via post-translational regulations in infected cells. It has been shown that ERK1/2 activation stabilizes c-Myc via serine phosphorylation at amino acid (a.a.) residue 62 of c-Myc (Amati, 2004; Laurenti et al., 2009). On the other hand, the activation of glycogen synthase kinase (GSK) causes threonine phosphorylation at a.a. 58 of c-Myc, leading to serine dephosphorylation at a.a. 62, which results in Fbw7-mediated poly-ubiquitination and c-Myc degradation (Amati, 2004; Laurenti et al., 2009). Studies by the present inventors and others have shown that pathogenic infection (such as mycobacteria infection) stimulates ERK1/2 and GSK3β expression (Chan et al., 2009; Cheung et al., 2005; Cheung et al., 2009; Song et al., 2003; Yadav and Schorey, 2006). It is thus postulated that the induction of c-Myc protein during infection is regulated by ERK1/2 activation and other mechanisms.

In addition, it is now discovered that the induction of c-Myc during infection (such as mycobacteria infection) selectively augments the induction of pro-inflammatory cytokines such as TNF-α and IL-6 in primary human macrophages. It is also discovered that c-Myc regulates pro-inflammatory cytokine responses by activating the IRAK1-dependent pathway. On the other hand, it is discovered that c-Myc does not regulate anti-inflammatory pathways, and does not stimulate the expression of anti-inflammatory cytokines such as IL10.

The selective induction of cytokine expression by c-Myc during infection is similar to the induction of the expression of early factors such as ATF3, CEBPδ and XBP1 by TLRs, which fine-tune the downstream innate immune responses via negative or positive feedback controls (Litvak et al., 2009; Maranon et al., 2010; Thompson et al., 2009). It is postulated that c-Myc fine-tunes its downstream innate immune responses via positive feedback controls.

In addition, it is discovered that the induction of cytokine expression can be blocked, at least partially, by c-Myc inhibitors such as c-Myc siRNA. Nevertheless, it is observed that, in the subject invention, while c-Myc siRNA (with more than 99% transfection efficiency) was employed at a high concentration that in theory could completely inactivate c-Myc expression, c-Myc-induced expression of pro-inflammatory cytokines was not completely inactivated. One explanation is that the particular c-Myc siRNA used in the subject invention cannot completely inactivate c-Myc transcription, as similar studies have also suggested that this c-Myc siRNA only partially blocked c-Myc expression in human cancer cell lines (Gao et al., 2009). Another explanation is that when c-Myc expression is blocked, cytokine expression can still be induced by alternative signaling pathways, which produce low albeit significant expression of pro-inflammatory cytokines such as TNF-α and IL-6.

Further, it is discovered that c-Myc does not regulate or cause PBMac cell death during acute infection (such as acute mycobacteria infection). In addition, c-Myc does not directly affect mycobacterial survival during acute infection. In comparison, previous studies have shown that c-Myc induces apoptosis or cell death in mouse macrophages infected with H. pylori and S. typhimurium, respectively (Cheng et al., 2005; Seong et al., 2009). Previous studies have also shown that c-Myc overexpression represses the interaction of IRF8 with Miz1 in mouse macrophages, resulting in the inhibition of NRAMP1 expression, and thereby suppressing the intracellular growth of S. enterica serovar typhimurium and BCG (Alter-Koltunoff et al., 2008; Bowen et al., 2002).

In addition, previous reports have demonstrated that c-Myc acts as a transcription factor to induce the mRNA expression of cytokines including IL-2, IL-13 and IL-17C in cancer cell lines (Fernandez et al., 2003; Li et al., 2003; Zeller et al., 2003). However, the role and mechanism of c-Myc in triggering innate immune responses in primary antigen-presenting cells have not been reported.

Surprisingly, it is now discovered that c-Myc acted as a cytoplasmic factor, rather than a transcription factor, for induction of cytokines in immune cells during infection. Although a previous report showed that c-Myc protein level increases in the nucleus upon S. typhimurium infection in mouse macrophages (Seong et al., 2009), several other studies have proved that c-Myc can localize in the cytoplasm in various cells including lymphoid, differentiated myeloid and HeLa cells (Craig et al., 1993; Niklinski et al., 2000; Peukert et al., 1997).

As is demonstrated in the Examples, c-Myc can colocalize with a-tubulin and reside in the cytoplasm of primary human macrophages. It is also postulated that c-Myc enhances IRAK1 degradation without directly interacting with MyD88 and IRAK1. Moreover, in contrast to a previous report (Fukunaga and Hunter, 1997), it is discovered that c-Myc did not interact with ERK1, but enhanced the activation of ERK1/2 and p38 kinases. Hence, it is postulated that c-Myc induces the activation of ERK1/2 as well as p38 kinases by promoting the upstream IRAK1 signaling.

Furthermore, it is discovered that cytoplasmic c-Myc decreased IκBα protein level and constitutively interacted with IκBα. These interactions may enhance IκBα protein degradation, leading to a continuous release and activation of NFκB for nuclear translocation. As a consequence of these c-Myc-stimulated signaling cascades, the induction of pro-inflammatory cytokines including TNF-α and IL-6 was augmented.

In contrast, cytoplasmic c-Myc only slightly inhibited the mycobacteria-activation of Akt and did not affect the mycobacteria-induction of IL-10. In line with our previous report (Chan et al., 2009), mycobacteria may utilize an alternative Akt signaling pathway for inducing anti-inflammatory cytokines such as IL-10. Thus, while c-Myc selectively enhances pro-inflammatory signaling pathways, it does not regulate anti-inflammatory pathways.

The expression of pro-inflammatory cytokines enhanced by c-Myc plays a crucial role in combat against mycobacteria. For instance, in conjunction with TGF-β, IL-6 has been suggested to initiate the differentiation of T_{H}17 cells, leading to the maintenance of granulomas and the expression of memory responses (Bettelli et al., 2008; Cooper and Khader, 2008). Additionally, previous in vivo studies have demonstrated that TNF-α produced from macrophages plays an important role in suppressing the intracellular growth of mycobacteria and in promoting the formation of granulomas to prevent mycobacterial dissemination (Ehlers, 2003; Flynn et al., 1995; Jacobs et al., 2000; Kaufmann and McMichael, 2005; Leemans et al., 2005; Russell, 2007; Teitelbaum et al., 1999). In addition, cytokines induced by TLR and its MyD88-IRAK1-dependent signaling pathway can lead to protection against not only mycobacteria (Bafica et al., 2005; Heldwein et al., 2003), but also other intracellular pathogens such as Listeria monocytogenes and Toxoplasma gondii (O'Neill and Bowie, 2007).

Thus, it is contemplated that cytoplasmic c-Myc modulates innate immunity via its role as a positive feedback control in the activation of pro-inflammatory pathways. As a result, modulating or increasing the level of c-Myc and modulating or enhancing its activity in the induction of pro-inflammatory pathways, in a controlled manner, can be used to enhance the host immune system function in controlling infection (such as viral, bacterial, fungal, microbial and other pathogenic infection), particularly acute infection. Modulating or increasing the level of c-Myc and modulating or enhancing its activity in the induction of pro-inflammatory pathways, in a controlled manner, can also be used to restore or improve compromised host immune system function in individuals. In addition, modulating or reducing the level of c-Myc and its activity in the induction of pro-inflammatory pathways, in a controlled manner, can be used to inhibit or suppress unwanted immune responses or autoimmune reactions, which are associated, at least in part, with increased or dysregulated cytoplasmic level of c-Myc or c-Myc associated pro-inflammatory signaling pathways.

Specifically, it is discovered that cytoplasmic c-Myc modulates pro-inflammatory immune responses during HIV infection. HIV dysregulates host immune responses, leading to increased production of pro-inflammatory cytokines such as TNF-α, IL-6, and IL-1β in immune cells (such as monocytes and macrophages). These pro-inflammatory cytokines activate NF-kB and NF-IL-6, which become translocated into the nucleus to act as transcription factors regulating the expression of a range of genes involved in immune responses. NF-kB and NF-IL-6 also bind to various enhancers of HIV long terminal repeats (LTR), resulting in increased transcription of HIV genome and increased retroviral replication (Li, Yim, and Lau A S, 2010). HIV infection also suppresses the activity of toll-like receptors such as TLR4 (Yim, Li, Lau J S and Lau A S, 2009). As a result, HIV-infected patients are susceptible to coinfecting opportunistic pathogens and may further develop HIV-associated disorders. It is contemplated that modulating the level or activity of cytoplasmic c-Myc during HIV infection, in a controlled manner, can be used to prevent, treat or ameliorate concurrent opportunistic infections and other HIV-associated disorders.

While in certain experimental models of the subject invention c-Myc expression and c-Myc associated pro-inflammatory signaling pathways were induced by mycobacteria infection, it would be readily understood that the benefits of the subject invention extend beyond the treatment of mycobacteria infection. For instance, those skilled in the art, benefited from the teachings of the subject invention, would recognize that the subject invention can be used to treat or ameliorate, or to develop therapeutics in the treatment or amelioration of, a variety of bacterial infections, such as infection by Staphylococcus aureus, Escherichii coli and Listeria monocytogenes.

Treatment of Inflammation and/or Immune Disorders

One aspect of the subject invention provides methods for treatment or amelioration of inflammatory diseases and/or immune disorders, particularly diseases or disorders associated with cytoplasmic c-Myc. The method comprises administering, to a subject in need of such treatment, an effective amount of an agent or a composition that inhibits or reduces c-Myc transcription/expression, c-Myc protein level in the cytoplasm, and/or its activity in regulating pro-inflammatory pathways. In an embodiment, the therapeutic agent or composition of the subject invention increases the level or activity of c-Myc antagonists including, but are not limited to, MAD1, MAX, MNT, MXD1-4 (formerly MAD1, MXI 1, MAD3 and MAD4) and MGA. Therapeutic agents of the subject invention can be a drug, chemical, compound, protein or peptide, or nucleic acid molecule (e.g. DNA, RNA such as siRNA).

Agents that inhibit c-Myc activity include, but are not limited to, agents that inhibit c-Myc transcription/expression; agents that inhibit c-Myc activity; and anti-c-Myc antibodies, aptamers, and c-Myc binding partners. In one embodiment, anti-c-Myc antibodies, aptamers, and c-Myc binding partners are used to reduce c-Myc level and/or c-Myc activity in the cytoplasm.

Agents that inhibit c-Myc transcription/expression include, but are not limited to, S2T1-6OTD, Quarfloxin (CX-3543), benzoylanthranilic acid, and Cationic Porphyrin TMPyP4. Specifically, Cationic Porphyrin TMPyP4 has been shown to inhibit both the expression and activity of c-Myc, and has been used in suppressing tumor proliferation. None of the c-Myc inhibitors, however, has been used for the treatment of inflammation and/or immune disorders.

Inhibitors of c-Myc transcription/expression also include antisense polynucleotides, such as short interfering nucleic acids (siNAs) that specifically target c-Myc mRNA. In an embodiment, the c-Myc inhibitor is an antisense polynucleotide that targets human c-Myc mRNA. In some embodiments, the c-Myc antisense polynucleotides target c-Myc mRNAs of non-human animals including, but not limited to, apes, chimpanzees, orangutans, monkeys, dogs, cats, horses, cattle, pigs, sheep, goats, chickens, mice, rats, and guinea pigs. The skilled artisan would readily appreciate that the antisense polynucleotides can be designed to target any c-Myc mRNAs publically known.

In certain specific embodiments, c-Myc siRNAs include, for example, SEQ ID NO:1, SEQ ID NO:2, SEQ ID NO:3, SEQ ID NO:4 (siGENOME SMARTpool L-003282-00 is a mixture of SEQ ID NOs:1-4), SEQ ID NOs:5-6 (SEQ ID NOs: 609, 613 of U.S. Pat. No. 7,659,389), SEQ ID NO:7 (SEQ ID NO:1 of U.S. Pat. No. 6,875,747), SEQ ID NOs: 8-11 (SEQ ID NOs: 1-4 of U.S. Pat. No. 6,544,755), and SEQ ID NOs 12-14 (SEQ ID NOs: 1, 7, 10 of U.S. Pat. No. 6,159,946).

Agents that inhibit the formation of c-Myc/Max heterodimeric transcription factor include, for example, 10058-F4,(Z,E)-5-(4-Ethylbenzylidine)-2-thioxothiazolidin-4-one.

Agents that inhibit the formation of c-Myc/Max heterodimeric transcription factor further include, but are not limited to,

a) bicyclo[2.2.1]hept-2-yl-[2-(4-nitro-phenyl)-ethyl]-amine;

b) 4-Methyl-2-[N′-(6-methyl-2-phenyl-chroman-4-ylidene)-hydrazino]-thiazole-5-carboxylic acid(3-nitro-phenyl)-amide;

c) 5-(4-Ethyl-benzylidene)-2-thioxo-thiazolidin-4-one;

d) 3-[3-(3,6-Dichloro-carbazol-9-yl)-2-hydroxy-propyl]-thiazolidine-2,4-dion-e;

e) biphenyl-2-yl-(7-nitro-benzo[1,2,5]oxadiazol-4-yl)-amine;

f) 1-(3-Chloro-phenyl)-3-diethylamino-pyrrolidine-2,5-dione; and

g) 1-[2,5-dioxo-1-(4-propoxy-phenyl)-pyrrolidin-3-yl]-piperidine-4-carboxyli-c acid.

In one embodiment, the c-Myc inhibitor is an antibody that binds to c-Myc. In a specific embodiment, the c-Myc inhibitor is an antibody that binds specifically to c-Myc. In a further specific embodiment, the c-Myc inhibitor is an antibody that binds specifically to human c-Myc. In a further specific embodiment, the c-Myc inhibitor is an antibody that binds specifically to a human c-Myc of SEQ ID NO:15 (GenBank Accession No. P01106). In certain embodiments, the c-Myc inhibitor is an antibody that binds specifically to a c-Myc protein comprising an amino acid sequence having at least 70%, 75%, 80%, 85%, 90%, 95%, 97%, or 99% percent identity to SEQ ID NO:15.

Unless otherwise specified, as used herein percent sequence identity and/or similarity of two sequences can be determined using the algorithm of Karlin and Altschul (1990), modified as in Karlin and Altschul (1993). Such an algorithm is incorporated into the NBLAST and XBLAST programs of Altschul et al. (1990). BLAST searches can be performed with the NBLAST program, score=100, wordlength=12, to obtain sequences with the desired percent sequence identity. To obtain gapped alignments for comparison purposes, Gapped BLAST can be used as described in Altschul et al. (1997). When utilizing BLAST and Gapped BLAST programs, the default parameters of the respective programs (NBLAST and XBLAST) can be used. See NCBI/NIH website.

“Specific binding” or “specificity” refers to the ability of a protein to detectably bind an epitope presented on a protein or polypeptide molecule of interest, while having relatively little detectable reactivity with other proteins or structures. Specificity can be relatively determined by binding or competitive binding assays, using, e.g., Biacore instruments. Specificity can be exhibited by, e.g., an about 10:1, about 20:1, about 50:1, about 100:1, 10.000:1 or greater ratio of affinity/avidity in binding to the specific target molecule versus nonspecific binding to other irrelevant molecules.

Anti-c-Myc antibodies of the present invention can be in any of a variety of forms, including intact immunoglobulin molecules, fragments of immunoglobulin molecules such as Fv, Fab and similar fragments; multimers of immunoglobulin molecules (e.g., diabodies, triabodies, and bi-specific and tri-specific antibodies, as are known in the art; see, e.g., Hudson and Kortt, J. Immunol. Methods 231:177 189, 1999); fusion constructs containing an antibody or antibody fragment; and human or humanized immunoglobulin molecules or fragments thereof.

Antibodies within the scope of the invention can be of any isotype, including IgG, IgA, IgE, IgD, and IgM. IgG isotype antibodies can be further subdivided into IgG1, IgG2, IgG3, and IgG4 subtypes. IgA antibodies can be further subdivided into IgA1 and IgA2 subtypes.

Antibodies of the present invention include polyclonal and monoclonal antibodies. The term “monoclonal antibody,” as used herein, refers to an antibody or antibody fragment obtained from a substantially homogeneous population of antibodies or antibody fragments (i.e. the individual antibodies within the population are identical except for possible naturally occurring mutations that may be present in a small subset of the antibody molecules). In a preferred embodiment, the subject invention reduces the level c-Myc in the cytoplasm of blood cells and/or immune cells. In another preferred embodiment, the subject invention reduces cytoplasmic c-Myc activity, thereby enhancing pro-inflammatory responses. In certain embodiments, the subject invention can be used to reduce the level of one or more pro-inflammatory mediators including, but not limited to, TNF-α, IL-6, IL-2, IL-1β, and TGF-β.

In an embodiment, the subject invention can be used to inhibit, suppress or reduce IRAK1 degradation. In another embodiment, the subject invention can be used to inhibit, suppress, or reduce the phosphorylation and activation of ERK1/2 and/or p38 MAPK. In another embodiment, the subject invention can be used to inhibit, suppress or reduce the nuclear translocation of NFκB p65. In another embodiment, the subject invention can be used to inhibit, suppress or reduce binding of c-Myc to IκBα.

In an embodiment, the subject invention does not affect or reduce the level of anti-inflammatory mediators, such as IL-10. In an embodiment, the subject invention does not affect (i.e., does not promote or suppress) the phosphorylation or activation of Akt. In an embodiment, the subject invention does not affect the level or activity of c-Myc in the nucleus. In an embodiment, the subject invention does not inhibit the formation and/or activity of c-Myc/Max heterodimeric transcription factor.

In another embodiment, the method does not comprise administering agents that inhibit or reduces c-Myc expression; agents that inhibit c-Myc-mediated gene transcription; and/or agents that inhibit binding of c-Myc to Max to form c-Myc/Max heterodimeric transcription factor.

The term “treatment” or any grammatical variation thereof (e.g., treat, treating, and treatment etc.), as used herein, includes but is not limited to, ameliorating or alleviating a symptom of a disease or condition, reducing, suppressing, inhibiting, lessening, or affecting the progression, severity, and/or scope of a condition.

The term “effective amount,” as used herein, refers to an amount that is capable of treating or ameliorating a disease or condition or otherwise capable of producing an intended therapeutic effect. In certain embodiments, the effective amount enables a 5%, 10%, 20%, 30%, 50%, 60%, 70%, 80%, 90%, 95%, 99%, or 100% reduction in cytoplasmic c-Myc levels. In certain embodiments, the effective amount enables a 5%, 10%, 20%, 30%, 50%, 60%, 70%, 80%, 90%, 95%, 99%, or 100% reduction in the increase of pro-inflammatory responses induced by cytoplasmic c-Myc . In one specific embodiment, the effective amount reduces of binding of c-Myc to IκBα by 5%, 10%, 20%, 30%, 50%, 60%, 70%, 80%, 90%, 95%, 99%, or 100%.

Additionally or alternatively, the effective amount enables a 5%, 10%, 20%, 30%, 50%, 60%, 70%, 80%, 90%, 95%, 99%, or 100% reduction in the level of one or more pro-inflammatory cytokines, such as TNF-α, IL-6, IL-2, and TGF-β. Additionally or alternatively, the effective amount enables a 5%, 10%, 20%, 30%, 50%, 60%, 70%, 80%, 90%, 95%, 99%, or 100% reduction in the level of phosphorylated ERK1/2 and/or phosphorylated p38. Additionally or alternatively, the effective amount enables a 5%, 10%, 20%, 30%, 50%, 60%, 70%, 80%, 90%, 95%, 99%, or 100% reduction in the level of nucleus/cytoplasm NFκB p65 intensity ratio.

The term “subject,” as used herein, describes an organism, including mammals such as primates, to which treatment with the compositions according to the present invention can be provided. Mammalian species that can benefit from the disclosed methods of treatment include, but are not limited to, apes, chimpanzees, orangutans, humans, monkeys; domesticated animals such as dogs, cats, horses, cattle, pigs, sheep, goats, chickens; and other animals such as mice, rats, guinea pigs, and hamsters.

The subject invention can be used in the treatment or amelioration of inflammatory symptoms in any disease, condition or disorder, where suppression of inflammation or unwanted immune or autoimmune reaction is beneficial. In certain embodiments, the subject invention is useful to treat or ameliorate diseases or conditions including, but not limited to, infection, inflammation, allergenic reactions, diseases associated with cell proliferation, neoangiogenesis, malignancy, and cardiovascular diseases.

In certain embodiments, the subject invention is useful to treat or ameliorate inflammatory diseases including, but not limited to, rheumatoid arthritis, ulcerative colitis,

Behcet's diseases, Crohn's disease, chronic inflammatory bowel diseases, graft-versus-host and transplant rejection, and related disorders of the joints or musculoskeletal system in which suppression of unwanted immune reaction and/or inflammation is beneficial.

In certain embodiments, the subject invention is useful to treat or ameliorate a disease or condition associated with inflammation or immune disorders. The diseases or conditions include, but are not limited to, asthma, rhinitis, chronic obstructive pulmonary disease (COPD), chronic urticaria, atopic dermatitis, paranasal sinus disease, migraine, pancreatitis, cancer, and atherosclerosis.

In certain embodiments, the subject invention is useful to treat or ameliorate inflammation associated with pathogenic infection, including viral, bacterial, fungal, protozoan, and other microbial infection. In a specific embodiment, the subject invention is useful to treat or ameliorate inflammation associated with mycobateria infection (such as tuberculosis), especially acute stage of mycobacteria infection (such as tuberculosis).

In certain embodiments, the subject invention is useful to treat or ameliorate inflammation associated with bacterial infection, such as infection by mycobacteria (such as M. tuberculosis and M. avium intracellulaire), Candida (such as C. albicans and C. parvum), Staphylococcus aureus, Salmonella, Escherichii coli, Listeria monocytogenes, and L. amazonensis. In certain embodiments, the subject invention is useful to treat or ameliorate inflammation associated with infection by HIV, herpes simplex virus (HSV) including HSV-1 and HSV-2, human papillomavirus (HPV) (such as HPV-6, HPV-11, HPV-16, and HPV-18), respiratory syncytial virus, rhinovirus, hepatitis viruses including hepatitis A virus, hepatitis B virus, hepatitis C virus, hepatitis D virus, hepatitis E virus, hepatitis F virus, and hepatitis G virus, oncoviruses, human T-lymphotropic virus Type I (HTLV-1), bovine leukemia virus (BLV), and Epstein-Barr virus.

In addition, the subject invention is useful to treat or ameliorate HIV infection, opportunistic infection concurrent with HIV infection, and/or AIDS-associated disorders. In an embodiment, the subject invention is useful to treat or ameliorate inflammation or infection associated with the progression of HIV infection. In certain embodiments, the subject invention is useful to treat or ameliorate concurrent viral, bacterial, fungal, protozoan, and other microbial infections including, but not limited to, concurrent infection by mycobacteria (such as M. tuberculosis and M. avium intracellulaire), Candida (such as C. albicans and C. parvum), Staphylococcus aureus, Salmonella, Escherichii coli, Listeria monocytogenes, Listeria amazonensis, HSV including HSV-1 and HSV-2, KSHV, HPV including HPV-6, HPV-11, HPV-16, and HPV-18, respiratory syncytial virus, rhinovirus, hepatitis viruses including hepatitis A virus, hepatitis B virus, hepatitis C virus, hepatitis D virus, hepatitis E virus, hepatitis F virus, and hepatitis G virus, oncoviruses, human T-lymphotropic virus Type I (HTLV-1), bovine leukemia virus (BLV), and Epstein-Barr virus.

In addition, the subject invention is useful to treat or ameliorate HIV-associated disorders including, but not limited to, AIDS-Kaposi's sarcoma, lymphoma, AIDS-associated neurological disorders, AIDS-associated cholangiopathy, and AIDS-associated sclerosing.

The subject invention is particularly useful for treatment or amelioration of pulmonary and respiratory diseases, such as for example, sleep-disorder breathing, chronic obstructive pulmonary disease (COPD), obstructive sleep apnea (OSA), pneumonitis following prior infection, interstitial lung disease, idiopathic pulmonary fibrosis, and cystic fibrosis.

In addition, the subject invention is useful to treat or ameliorate allergy and/or inflammation accompanying allergenic diseases of respiratory, gastrointestinal and dermatological systems; and adipose tissue inflammation. The subject invention can also be used to treat or ameliorate inflammation associated with hypersensitivity, allergic reactions, asthma, and systemic lupus erythematosus; collagen diseases and autoimmune diseases, conditions or disorders in which immune and/or inflammation suppression is beneficial.

In a specific embodiment, the subject invention is useful for suppressing the metastatic spread of tumors that utilize TNF-α to evade immune surveillance and promote metastasis. Specifically, it is discovered by the present inventors that TNF-α induces the production of matrix metalloproteinases (MMPs) and MMP family members, which degrade extracellular matrix proteins. However, certain tumor cells (such as glioblastomas, lung carcinomas, breast carcinomas and nasopharyngeal carcinomas) have developed resistance to the cytotoxic effects of TNF-α. As a result, these tumor cells utilize the induction of MMP by TNF-α to invade neighboring tissues as well as organs located in distant parts of the body. In an embodiment, the subject invention can be used to treat or ameliorate glioblastoma multiforme, lung carcinoma, breast carcinoma, and nasopharyngeal carcinoma.

Additionally, the subject invention is useful to treat or ameliorate cerebrovascular and cardiovascular diseases, such as for example, angina, acute myocardial infarction, stroke, atherosclerosis, thrombosis, coronary angioplasty, aortic aneurysms, vascular inflammation, intimal hyperplasia, and hyperlipidemia-dependent aortic aneurysm.

In addition, the subject invention is useful to treat or ameliorate inflammation induced by, or associated with, diseases or conditions including, but not limited to, renal insufficiency arising from ischaemia, renal insufficiency induced by immunological reactions or chemicals (such as cyclosporin), migraine, cluster headache, ocular conditions such as uveitis; hepatitis (such as caused by chemicals, immunological reactions, and infection), trauma or shock (such as burn, injury and endotoxemia), and allograft transplant rejection.

Moreover, the subject invention is useful for treating or ameliorating inflammation associated with atherosclerosis, atherosclerotic heart disease; reperfusion injury, cardiac arrest, myocardial infarction, vascular inflammatory disorders including cerebrovascular diseases (such as stroke), respiratory distress syndrome and other cardiopulmonary diseases, conditions or disorders where suppression of unwanted immune responses and/or inflammation is beneficial.

In addition, the subject invention can be used to treat or ameliorate conditions associated with, or arising from, inflammation including, but not limited to, hepatic diseases such as hepatic fibrosis and liver cirrhosis, glandular diseases such as thyroiditis, and renal and urologic diseases including glomerulonephritis.

The subject invention can be used to treat or ameliorate inflammation associated with, or arising from, post-traumatic inflammation; septic shock; bacterial, viral (such as HIV), fungal and other microbial infection; surgery, transplantation (such as bone marrow transplantation), and gene therapy.

Further, the subject invention can also be used to inhibit macrophage or T cell associated aspects of an immune response not associated with inflammation. The subject invention can be used to inhibit macrophage or T cell activities including, but not limited to, macrophage antigen-presenting activity, macrophage-associated cytokine production, T cell cytokine production, T cell adhesion, and T cell proliferative activities. Thus, the subject invention is also useful to suppress or inhibit humoral and/or cellular immune responses.

The subject invention is also useful to treat or ameliorate monocyte and leukocyte proliferative diseases, e.g., leukemia, by reducing the level of monocytes and lymphocytes.

The subject invention is further useful for the prevention and/or treatment of graft rejection in cases of transplantation of natural or artificial cells, tissue and organs, such as cornea, bone marrow, organs, lenses, pacemakers, natural and artificial skin tissue, and the like.

The subject invention is also useful to treat or ameliorate inflammation associated with otitis and other otorhinolaryngological diseases, conditions or disorders where inflammation and/or immune suppression is beneficial; dermatitis and other dermal diseases, conditions or disorders where inflammation and/or immune suppression is beneficial; and periodontal diseases and other dental diseases, conditions or disorders where inflammation and/or immune suppression is beneficial.

In addition, the subject invention is also useful to treat or ameliorate inflammation including, but not limited to, conditions associated with ophthalmologic diseases such as posterior uveitis; intermediate uveitis, anterior uveitis, conjunctivitis, chorioretinitis, uveoretinitis, optic neuritis, intraocular inflammation such as retinitis, scleritis, retinitis pigmentosa, immune and inflammatory components of degenerative fondus disease, inflammatory components of ocular trauma, proliferative vitreoretinopathies, acute ischemic optic neuropathy, and post-surgical excessive scarring.

Moreover, the subject invention is also useful to treat or ameliorate inflammation associated with autoimmune diseases both in the central nervous system (CNS) and in any other organ. These diseases or conditions include, but are not limited to, Parkinson's disease, complications and/or side effects from treatment of Parkinson's disease, AIDS-related dementia complex, Devic's disease, Sydenham chorea, Alzheimer's disease and other degenerative diseases, conditions or disorders of the central nervous system where immune and/or inflammation suppression is beneficial.

The subject invention is also useful to treat or ameliorate strokes (cerebrovascular diseases), post-polio syndrome, immune and inflammatory components of psychiatric disorders, myelitis, encephalitis, subacute sclerosing panencephalitis, encephalomyelitis, acute neuropathy, subacute neuropathy, chronic neuropathy, Guillain-Barre syndrome, Sydenham chorea, myasthenia gravis, Huntington's disease; amyotrophic lateral sclerosis, CNS infections or hypoxia-ischemia, and inflammatory aspects of muscular atrophies and dystrophies.

### Drug Screening Assays for Treatment of Inflammation and Immune Disorders

The subject invention also provides methods for screening for therapeutic agents for treatment or amelioration of inflammation or immune diseases or disorders, by selecting agents that reduce the level of c-Myc in the cytoplasm and/or inhibit or reduce the induction of pro-inflammatory responses by c-Myc. The therapeutic agents can be a drug, chemical, compound, protein or peptide, or nucleic acid molecule (e.g. DNA, RNA such as siRNA).

Any c-Myc proteins or peptides that are publically known can be used to perform the screening assay of the subject invention. In a preferred embodiment, human c-Myc protein is used. In a specific embodiment, the human c-Myc protein comprises SEQ ID NO:15. In certain embodiments, the c-Myc protein useful for performing the screening assay comprises an amino acid sequence having at least 70%, 75%, 80%, 85%, 90%, 95%, 97%, or 99% percent identity to SEQ ID NO:15.

Specifically, the subject methods can be used for screening for therapeutic agents that inhibit or reduce c-Myc transcription/expression, reduce c-Myc mRNA and/or protein stability, or otherwise reduce the level of c-Myc protein in the cytoplasm. In an embodiment, the subject screening method comprises:

a) contacting a candidate agent with cells expressing c-Myc;

b) determining a level of c-Myc expression in the cells; and

c) selecting the candidate agent if said agent reduces the level of c-Myc expression.

In an embodiment, agents that increase the level or activity of c-Myc antagonists are selected. c-Myc antagonists include, but are not limited to, MAD1, MAX, MNT, MXD1-4 (formerly MAD1, MXI1, MAD3 and MAD4) and MGA. See Hurlin et al., 2006; Zhu et al., 2008.

In a specific embodiment, the method further comprises, after step a): determining a level or activity of a c-Myc antagonist selected from MAD 1, MAX, MNT, MXD1-4 or MGA; and selecting the candidate agent if said agent increases the level or activity of the c-Myc antagonist.

The level of c-Myc expression can be determined at nucleic acid levels (such as mRNA) or protein levels. In a preferred embodiment, the level of c-Myc protein in the cytoplasm is determined, and the candidate agent that reduces the level of c-Myc protein in the cytoplasm is selected. The determination can be measured once, or at multiple times, after the addition of a candidate molecule.

In a specific embodiment, the subject screening method comprises:

contacting a candidate agent with cells expressing c-Myc;

determining a level of c-Myc protein in the cytoplasm of the cells;

determining a level of c-Myc expression; and

selecting the candidate agent if said agent reduces the level of c-Myc protein in the cytoplasm, but does not reduce c-Myc expression.

Preferred cells for the subject screening assays include blood cells, such as peripheral blood mononucleated cells (PBMC); immune cells, such as T cells, T helper cells, B cells, macrophages, leucocytes, lymphocytes, monocytes; and other cells capable of releasing immune modulators (e.g., c-Myc, pro-inflammatory mediators, and anti-inflammatory mediators). Preferably, cells used in the screening assay contain c-Myc proteins in the cytoplasm and do not contain c-Myc proteins in the nucleus. In an embodiment, c-Myc proteins are present in the cytoplasm and the nucleus of the cells.

In addition, the subject methods can be used for screening for therapeutic agents that inhibit or reduce the induction of pro-inflammatory responses by cytoplasmic c-Myc. In an embodiment, the subject screening method comprises:

a) contacting a candidate agent with cells expressing c-Myc and a pro-inflammatory mediator, wherein c-Myc is present in the cytoplasm of the cells;

b) determining a level of the pro-inflammatory mediator in the cells; and

c) selecting the candidate agent if said agent reduces the level of the pro-inflammatory mediator.

Suitable pro-inflammatory mediators include, but are not limited to, TNF-α, IL-6, IL-2, IL-1β, and TGF-β.

In another embodiment, the subject screening method comprises:

a) contacting a candidate agent with cells expressing c-Myc and IRAK1, wherein c-Myc is present in the cytoplasm of the cells;

b) determining a level of IRAK1 in the cells;

c) selecting the candidate agent if said agent reduces the level of IRAK1.

In another embodiment, the subject screening method comprises:

a) contacting a candidate agent with cells expressing c-Myc and ERK1/2 and/or p38, wherein c-Myc is present in the cytoplasm of the cells;

b) determining a level of phosphorylated ERK1/2 and/or phosphorylated p38 in the cells;

c) selecting the candidate agent if said agent reduces the level of phosphorylated ERK1/2 and/or phosphorylated p38.

In another embodiment, the subject screening method comprises:

a) contacting a candidate agent with cells expressing c-Myc and IκBα, wherein c-Myc is present in the cytoplasm of the cells;

b) determining a level of IκBα in the cells;

c) selecting the candidate agent if said agent reduces the level of IκBα.

In another embodiment, the subject screening method comprises:

a) contacting a candidate agent with cells expressing c-Myc, wherein the c-Myc is present in the cytoplasm;

b) determining binding of c-Myc to IκBα; and

c) selecting the candidate agent if said agent inhibits or reduces binding of c-Myc to IκBα. Optionally, the method further comprises: determining nucleus/cytoplasm NFκB p65 intensity ratio in the cells; and selecting the candidate agent if said agent reduces the nucleus/cytoplasm NFκB p65 intensity ratio.

In a further embodiment, the candidate therapeutic agent is selected if it does not reduce the levels of anti-inflammatory mediators, such as IL-10, in the cells. In a further embodiment, the candidate therapeutic agent is selected if it does not inhibit or suppress the activation of kinsases that activate anti-inflammatory pathways. In an embodiment, the candidate therapeutic agent is selected if it does not inhibit or suppress the phosphorylation of Akt in the cells.

In a specific embodiment, the subject screening assays can be used for developing oligonucleotides in antisense therapy. Candidate antisense therapeutic agents include oligonucleotide molecules (DNA or RNA oligonucleotides) that specifically hybridize (e.g. bind), via complementary base pairing under cellular conditions, with the mRNA and/or genomic DNA encoding c-Myc protein so that transcription and/or translation of c-Myc protein can be blocked or disrupted. The DNA-RNA or RNA-RNA can also serve as a substrate for the cellular enzyme ribonuclease H, which destroys the integrity of mRNA.

Absolute complementarity, although preferred, is not required. A sequence “complementary” to a portion of an RNA, as referred to herein, means a sequence having sufficient complementarity to be able to hybridize with the RNA, forming a stable duplex. In the case of double-stranded antisense nucleic acids, a single strand of the duplex DNA may thus be tested, or triplex formation may be assayed. The ability to hybridize will depend on both the degree of complementarity and the length of the antisense nucleic acid. One skilled in the art would also ensure that the length of the antisense oligonucleotides is long enough to avoid chance complementarity to points on untargeted mRNAs.

In a further embodiment, the subject screening methods can be used to generate c-Myc mimetics or inhibitors, e.g. peptide or non-peptide agents, which are capable of disrupting binding of the c-Myc with IκBα or other proteins that enhance pro-inflammatory responses, to inhibit or suppress pro-inflammatory responses.

The subject drug screening assays can be performed using techniques well known in the art, including but not limited to, Western blots, Northern blots, Southern blots, enzyme-linked immunosorbent assay (ELISA), polymerase chain reaction (PCR), immunoprecipitation, immunofluorescence, radioimmunoassay, immunocytochemistry, nucleic acid hybridization techniques, nucleic acid reverse transcription methods, and nucleic acid amplification methods.

Further, the subject screening assays can be performed in vitro or in vivo. For instance, the therapeutic agent may be administered to a subject (such as mice) to further examine the therapeutic effect on suppressing inflammation and/or unwanted immune responses in vivo.

The subject screening assays can be used to develop therapeutics in the treatment or amelioration of inflammatory symptoms in any disease, condition or disorder, where reduction of c-Myc level in the cytoplasm and/or inhibition or suppression of c-Myc-induced pro-inflammatory responses is beneficial. In certain embodiments, the subject screening assays can be used to develop therapeutics for the treatment or amelioration of diseases or conditions including, but not limited to, inflammation, allergenic reactions, diseases associated with cell proliferation, neoangiogenesis, malignancy, and cardiovascular diseases. In preferred embodiments, the subject screening assays can be used to develop therapeutics for the treatment of inflammatory diseases including, but not limited to, multiple sclerosis, ulcerative colitis, rheumatoid arthritis and systemic lupus.

In certain embodiments, the subject screening assays can be used to develop therapeutics for the treatment or amelioration of HIV infection, opportunistic infection concurrent with HIV infection, and AIDS-associated disorders. In certain embodiments, the subject screening assays can be used to develop therapeutics for the treatment or amelioration of concurrent viral, bacterial, fungal, protozoan, and other microbial infections including, but not limited to, concurrent infection by mycobacteria (such as M. tuberculosis and M. avium intracellulaire), Candida (such as C. albicans and C. parvum), Staphylococcus aureus, Salmonella, Escherichii coli, Listeria monocytogenes, Listeria amazonensis, HSV including HSV-1 and HSV-2, KSHV, HPV including HPV-6, HPV-11, HPV-16, and HPV-18, respiratory syncytial virus, rhinovirus, hepatitis viruses including hepatitis A virus, hepatitis B virus, hepatitis C virus, hepatitis D virus, hepatitis E virus, hepatitis F virus, and hepatitis G virus, oncoviruses, human T-lymphotropic virus Type I (HTLV-1), bovine leukemia virus (BLV), and Epstein-Barr virus.

In certain embodiments, the subject screening assays can be used to develop therapeutics for the treatment or amelioration of HIV-associated disorders including, but not limited to, AIDS-Kaposi's sarcoma, lymphoma, AIDS-associated neurological disorders, AIDS-associated cholangiopathy, and AIDS-associated sclerosing.

In another embodiment, the subject invention provides a method for enhancing pro-inflammatory immune activity, comprising administering an effective amount of a c-Myc protein and/or a nucleic acid encoding a c-Myc protein.

### Drug Screening Assays for Enhancing Host Immune Responses

The subject invention also provides methods for screening for therapeutic agents for enhancing host immune responses for controlling pathogenic infection and/or for restoring or improving compromised immune system function. The method selects agents that increase the level of c-Myc in the cytoplasm and/or enhance the induction of pro-inflammatory responses by c-Myc. The therapeutic agent can be a drug, chemical, compound, protein or peptide, or nucleic acid molecule (e.g. DNA, RNA).

Any c-Myc proteins or peptides that are publically known can be used to perform the screening assay of the subject invention. In a preferred embodiment, human c-Myc protein is used. In a specific embodiment, the human c-Myc protein comprises SEQ ID NO:15. In certain embodiments, the c-Myc protein useful for performing the screening assay comprises an amino acid sequence having at least 70%, 75%, 80%, 85%, 90%, 95%, 97%, or 99% percent identity to SEQ ID NO:15.

In an embodiment, the subject invention provides methods for developing therapeutic agents for the treatment or amelioration of bacterial, viral, microbial and/or other pathogenic infection. In a specific embodiment, the subject invention provides methods for developing therapeutic agents for the treatment or amelioration of infection, especially acute infection, by human immunodeficiency virus (HIV), mycobacteria (such as Mycobacterium tuberculosis, Mycobacterium avium, M. bovis (BCG), M. kansasii and M. chelonae), influenza, and hepatitis.

In an embodiment, the subject screening method comprises:

a) contacting a candidate agent with cells expressing c-Myc;

b) determining a level of c-Myc expression in the cells; and

c) selecting the candidate agent if said agent increases the level of c-Myc expression.

In a preferred embodiment, a level of c-Myc protein in the cytoplasm is determined, and the candidate agent that increases the level of c-Myc protein in the cytoplasm is selected.

In addition, the subject methods can be used for screening for therapeutic agents that enhance the induction of pro-inflammatory responses by c-Myc. In an embodiment, the subject screening method comprises:

a) contacting a candidate agent with cells expressing c-Myc and a pro-inflammatory mediator, wherein c-Myc is present in the cytoplasm of the cells;

b) determining a level of the pro-inflammatory mediator in the cells; and

c) selecting the candidate agent if said agent increases the level of the pro-inflammatory mediator.

Suitable pro-inflammatory mediators include, but are not limited to, TNF-α, IL-6, IL-2, IL-1β, and TGF-β.

In another embodiment, the subject screening method comprises:

a) contacting a candidate agent with cells expressing c-Myc and IRAK1, wherein c-Myc is present in the cytoplasm of the cells;

b) determining a level of IRAK1 in the cells;

c) selecting the candidate agent if said agent increases the level of IRAK1.

In another embodiment, the subject screening method comprises:

a) contacting a candidate agent with cells expressing c-Myc and ERK1/2 and/or p38, wherein c-Myc is present in the cytoplasm of the cells;

b) determining a level of phosphorylated ERK1/2 and/or phosphorylated p38 in the cells;

c) selecting the candidate agent if said agent increases the level of phosphorylated ERK1/2 and/or phosphorylated p38.

In another embodiment, the subject screening method comprises:

a) contacting a candidate agent with cells expressing c-Myc and IκBα, wherein c-Myc is present in the cytoplasm of the cells;

b) determining a level of IκBα in the cells;

c) selecting the candidate agent if said agent increases the level of IκBα.

In another embodiment, the subject screening method comprises:

a) contacting a candidate agent with cells expressing c-Myc and IκBα, wherein c-Myc is present in the cytoplasm of the cells;

b) determining a level of binding of c-Myc to IκBα in the cells;

c) selecting the candidate agent if said agent enhances binding of c-Myc to IκBα.Optionally, the method further comprises: determining nucleus/cytoplasm NFκB p65 intensity ratio in the cells; and selecting the candidate agent if said agent increases the nucleus/cytoplasm NFκB p65 intensity ratio.

Further, the subject screening assays can be performed in vitro or in vivo. For instance, the therapeutic agent may be administered to a subject (such as mice) to further examine the therapeutic effect on enhancing immune responses in vivo.

In a specific embodiment, the subject invention provides methods for developing therapeutic agents for the treatment or amelioration mycobateria infection (such as tuberculosis), especially acute stage of mycobacteria infection (such as tuberculosis). In certain embodiments, the subject invention provides methods for developing therapeutic agents for the treatment or amelioration bacterial infections, such as infection by Staphylococcus aureus, Escherichii coli and Listeria monocytogenes.

### Therapeutic Compositions and Formulations

The subject invention provides for therapeutic or pharmaceutical compositions. In an embodiment, the composition comprises a therapeutically effective amount of a c-Myc inhibitor of the subject invention and, optionally, a pharmaceutically acceptable carrier. c-Myc inhibitors include agents that inhibit or reduce c-Myc transcription/expression, c-Myc protein level in the cytoplasm, and/or its activity in regulating pro-inflammatory pathways. The term “carrier” refers to a diluent, adjuvant, excipient, or vehicle with which the compound is administered. Such pharmaceutical carriers can be sterile liquids, such as water and oils, including those of petroleum oil such as mineral oil, vegetable oil such as peanut oil, soybean oil, and sesame oil, animal oil, or oil of synthetic origin. Saline solutions and aqueous dextrose and glycerol solutions can also be employed as liquid carriers, particularly for injectable solutions.

Suitable pharmaceutical excipients include starch, glucose, lactose, sucrose, gelatin, malt, rice, flour, chalk, silica gel, sodium stearate, glycerol monostearate, talc, sodium chloride, dried skim milk, glycerol, propylene, glycol, water, ethanol and the like. The therapeutic composition, if desired, can also contain minor amounts of wetting or emulsifying agents, or pH buffering agents. These compositions can take the form of solutions, suspensions, emulsion, tablets, capsules, granules, powders, sustained-release formulations and the like. The composition can be formulated with traditional binders and carriers such as triglycerides. Examples of suitable pharmaceutical carriers are described in “Remington's Pharmaceutical Sciences” by E. W. Martin. Such compositions contain a therapeutically effective amount of the therapeutic composition, together with a suitable amount of carrier so as to provide the form for proper administration to the patient. The formulation should suit the mode of administration.

The therapeutic or pharmaceutical compositions of the invention can be formulated as neutral or salt forms. Pharmaceutically acceptable salts include those formed with free amino groups such as those derived from hydrochloric, phosphoric, acetic, oxalic, tartaric acids, etc., and those formed with free carboxyl groups such as those derived from sodium, potassium, ammonium, calcium, ferric hydroxides, isopropylamine, triethylamine, 2-ethylamino ethanol, histidine, procaine, etc.

The invention also provides a pharmaceutical pack or kit comprising one or more containers filled with one or more of the ingredients, e.g., compound, carrier, of the pharmaceutical compositions of the invention.

### Routes of Administration

The compounds and compositions of the subject invention can be administered to the subject being treated by standard routes, including oral, inhalation, or parenteral administration including intravenous, subcutaneous, topical, transdermal, intradermal, transmucosal, intraperitoneal, intramuscular, intracapsular, intraorbital, intracardiac, transtracheal, subcutaneous, subcuticular, intraarticular, subcapsular, subarachnoid, intraspinal, epidural and intrasternal injection, infusion, and electroporation, as well as co-administration as a component of any medical device or object to be inserted (temporarily or permanently) into a subject.

The amount of the therapeutic or pharmaceutical composition of the invention which is effective in the treatment of a particular disease, condition or disorder will depend on the route of administration, and the seriousness of the disease, condition or disorder, and should be decided according to the judgment of the practitioner and each patient's circumstances. In general, the dosage ranges from about 0.001 mg/kg to about 300 mg/kg.

For instance, suitable unit dosages may be between about 0.01 to about 300 mg, about 0.01 to about 200 mg, about 0.01 to about 100 mg, about 0.01 to about 50 mg, about 0.01 to about 30 mg, about 0.01 to about 20 mg, about 0.01 to about 10 mg, about 0.01 to about 5 mg, about 0.01 to about 3 mg about, 0.01 to about 1 mg, about 0.01 to about 0.5 mg, about 0.01 to about 0.3 mg, about 0.01 to about 0.1 mg about, 0.01 to about 0.05 mg, or about 0.01 to about 0.02 mg. Such a unit dose may be administered more than once a day, e.g. two or three times a day.

The amount of active ingredient that may be combined with the carrier materials to produce a single dosage form will vary, depending such as the type of the condition and the subject to be treated. In general, a therapeutic composition contains from about 5% to about 95% active ingredient (w/w). More specifically, a therapeutic composition contains from about 20% (w/w) to about 80% or about 30% to about 70% active ingredient (w/w).

Once improvement of the patient's condition has occurred, a maintenance dose is administered if necessary. Subsequently, the dosage or the frequency of administration, or both, may be reduced as a function of the symptoms to a level at which the improved condition is retained. When the symptoms have been alleviated to the desired level, treatment should cease. Patients may however require intermittent treatment on a long-term basis upon any recurrence of disease symptoms.

In addition, in vitro assays may optionally be employed to help identify optimal dosage ranges. The precise dose to be employed in the formulation will also depend on the route of administration, and the seriousness of the disease, condition or disorder, and should be decided according to the judgment of the practitioner and each patient's circumstances. Effective doses may be extrapolated from dose-response curves derived from in vitro or animal model test systems.

### Materials and Methods

Reagents

Inhibitors antagonizing p38 kinases (SB203580), ERK1/2 (PD98059), JNK (SP600159), and NFκB (CAPE) were purchased from Calbiochem (Calbiochem, USA) and diluted in dimethyl sulfoxide (DMSO) before their addition to the cells.

### Mycobacteria

Bacille Calmette-Guérin (BCG) vaccine (Danish strain 1331, Statens Serum Institut, Denmark) was reconstituted following the manufacturer's protocol. Clinical isolates of M. avium, M. chelonae and M. kansasii (Dr. P L Ho, Department of Microbiology, the University of Hong Kong) were grown in BD BACTEC™ Myco/F Lytic Medium (BD Bioscience, USA) at 37° C. The mycobacteria isolates were washed with and resuspended in phosphate-buffered saline (PBS) containing 10% glycerol and 0.05% Tween 80 (Sigma, USA), and stored as aliquots at −70° C. until use. The mycobacteria were quantified.

### Isolation of Primary Human Blood Macrophages

Human peripheral blood mononuclear cells (PBMC) isolated from blood samples of healthy blood donors were obtained from Hong Kong Red Cross Blood Transfusion Service. Primary blood macrophages were differentiated from CD14⁻ monocytes (>90% purity), which were isolated from blood samples by Ficoll-Paque (GE Healthcare, USA) density gradient centrifugation, and purified by the adherence method (Lee et al., 2005).

### Antibodies

Primary antibodies were purchased from Millipore for detecting c-Myc and IRAK1 (Millipore, USA), from Cell Signaling Technology for detecting ERK1/2, phospho-ERK1/2, p38 and phospho-p38 (Cell Signaling Technology, USA), and from Santa Cruz Biotechnology for detecting NFκB p65, actin, IκBα, α-tubulin, MyD88 and lamin B (Santa Cruz Biotechnology, USA). The density of the protein bands was quantified by ImageJ software (National Institute of Health, USA).

siRNA Transfection
c-Myc-specific siRNA (siGENOME SMARTpool, L-003282-00, which is a mixture of SEQ ID NOs:1-4) and control siRNA (siGenome, Non-Targeting siRNA #2, D-001210-02) were purchased from Thermo Fisher Scientific, USA. PBMac were transfected with control or c-Myc specific siRNA (100 nM) by 4 μl INTERFERin (Polyplus-Transfection Inc., France) as previously described (Chan et al., 2010), and incubated for 48 hours before the indicated treatment. The percentage of cells transfected with Block-iT Fluorecent Oligo (Invitrogen, USA) by Cellomics ArrayScan HCS VTI Reader (Thermo Fisher Scientific, USA) was determined, and the transfection efficiency was >99%.

### Reverse-Transcription Quantitative PCR

DNaseI-treated RNA was reverse-transcribed into cDNA by SuperScript II System (Invitrogen, USA). The cDNA was subject to quantitative polymerase chain reaction (PCR) by TaqMan gene-specific assays-on-demand reagent kits (Applied Biosystems, USA) to measure the expression levels of TNF-α, IL-6 and IL-10 relative to those of GAPDH as previously described (Cheng et al., 2009; Lee et al., 2005; Li and Lau, 2007; Yim et al., 2009).

### Immunoprecipitation and Western Blot

Total proteins (200 μg) were extracted from PBMac by IP buffer (10 mM Tris [pH7.4], 1 mM EDTA, 1 mM EGTA, 150 mM NaCl, 0.5% NP-40, 1% Triton X-100, inhibitor cocktails of proteases and phosphatases), and were incubated with 1μg primary antibodies against IκBα(Santa Cruz Biotechnology, USA) or c-Myc (Millipore, USA) for 2 hours at 4° C. and then with 20 μl Protein A/G agarose beads (Santa Cruz Biotechnology, USA) for 1 hour at 4° C. The immunoprecipitates were then washed 6 times with IP buffer. Cytoplasmic and nuclear proteins were extracted from PBMac as previously described (Cheung et al., 2005; Lee et al., 2005). Immunoprecipitated, cytoplasmic (50 μg) or nuclear proteins (10 μg) were analyzed by Western Blot as in our previous reports (Cheung et al., 2005; Lee et al., 2005).

### Immunocytochemistry

PBMac were fixed with 4% paraformaldehyde and permeabilized by 0.25% Triton-X 100. Following incubation with 1% bovine serum albumin in PBS for blocking, cells were stained with anti-c-Myc primary monoclonal antibody (Millipore, USA) and fluorescein isothiocyanate (FITC)-conjugated secondary antibodies (Millipore, USA), followed by co-labeling with primary antibodies against a-tubulin or NFκB p65 (Santa Cruz Biotechnology, USA) and rhodamine-conjugated secondary antibodies (Millipore, USA). Nuclei of cells were stained with 4,6-diamidine-2-phenylindole dihydrochloride (DAPI). Immunofluorescent images were captured from the Zeiss LSM 510 Confocal system (Carl Zeiss Microlmaging Inc.) and analyzed by ImageJ software with Pearson-Spearman correlation colocalization plug-in to quantify the colocalization relationship between the two confocal images as previously described (French et al., 2008; Pryor et al., 2008). For assaying nuclear translocation of NFκB p65, cells were stained with primary antibodies against NFκB p65 (Santa Cruz Biotechnology, USA) and DAPI as described above. The immunofluorescence of the labeled cells was measured by Cellomics ArrayScan HCS VTI Reader (Thermo Fisher Scientific, USA) as previously described (Yim et al., 2009).

### Enzyme-Linked Immunosorbent Assay (ELISA)

Levels of cytokine proteins in the PBMac supernatants were determined by the specific ELISA kits (R&D Systems, USA).

### Colony-Forming Unit Assay of Mycobacteria

The mycobacteria cell number in PBMac was measured by lysing the cells with 0.5% Triton-X100 in PBS. The mycobacteria were 10-fold serially diluted and plated on Middlebrook 7H10 (BD Bioscience, USA) agar plate supplemented with 10% Middlebrook OADC Enrichment (BD Bioscience, USA) and 0.5% glycerol for M. avium or Mueller-Hinton agar plate (Oxoid, UK) for M. chelonae and M. kansasii. Colonies formed were counted as colony-forming unit (CFU).

### MTT Assay

MTT assay was performed as reported (Cheng et al., 2007). Briefly, cells were incubated with 1 mg/ml MTT (2-[4,5-dimethylthiazol-2-yl]-2,5-di-phenyltetrazolium bromide) (Sigma, USA) for 2 hours at 37° C. The MTT formazan developed was dissolved in 100 μl isopropanol. The absorbance of the solution was quantified at 570 nm against 650 nm by a microplate reader (BioRad, USA).

### Statistical Analysis

Two-tailed and paired Student's t-test was used to analyze the data.

### EXAMPLES

Following are examples that illustrate procedures for practicing the invention. These examples should not be construed as limiting.

### Example 1

### Induction of C-Myc Expression During Mycobacteria Infection

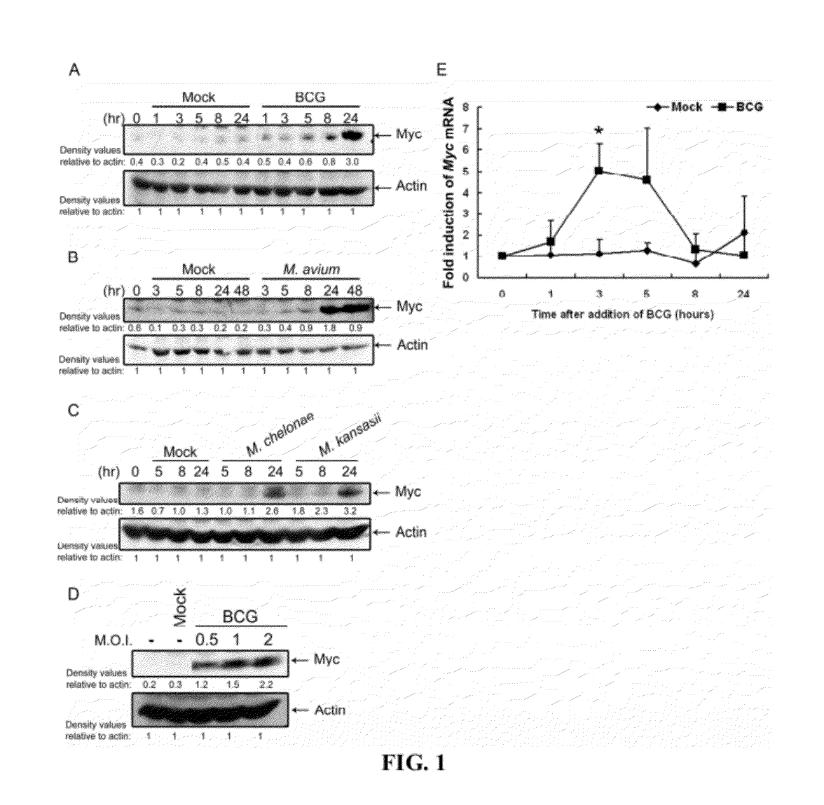

To examine whether mycobacteria induce c-Myc expression, primary human blood macrophages (PBMac) were treated with mycobacteria, including Bacillus Calmette-Guérin (BCG), M. avium, M. chelonae and M. kansasii. The results showed that c-Myc protein levels were increased in a time-dependent manner after exposure to mycobacteria, starting from 5 hour and reaching the highest point at 24 hour post-treatment (FIGS. 1A-C). Moreover, the level of c-Myc induction depends on the concentration of mycobacteria (FIG. 1D). In addition to the c-Myc expression at protein levels, c-Myc mRNA levels were significantly increased up to 5-fold by BCG in a time-dependent manner (FIG. 1E).

Example 2

### Induction of C-Myc Transcrption of by ERK1/2 and JNK1/2

To delineate the signaling mechanisms underlying mycobacteria induction of c-Myc expression, PBMac were pretreated with specific inhibitors against various signaling molecules or kinases for 1 hour, followed by BCG treatment for 3 hours. The level of BCG-induced c-Myc mRNA expression was measured by quantitative reverse-transcription-polymerase chain reaction (RT-PCR).

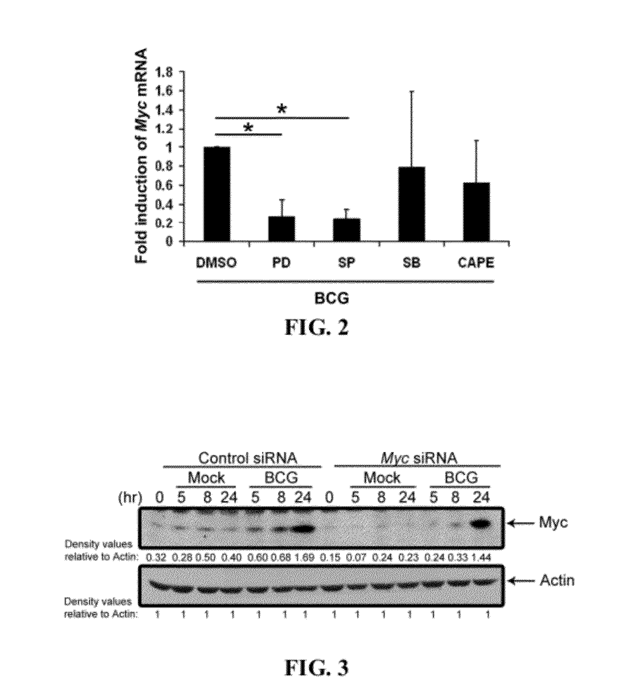

The results showed that the level of BCG-induced c-Myc mRNA expression was significantly reduced after pretreatment with inhibitors against ERK1/2 (PD98059) and JNK1/2 (SP600159), as compared to those treated with DMSO (FIG. 2). However, pretreatment with inhibitors against p38 kinases (SB203580) and NFκB (CAPE) did not affect the BCG-induced c-Myc mRNA transcription (FIG. 2). The results demonstrated that induction of c-Myc protein expression is a common pathway utilized by different species of mycobacteria. The induction of c-Myc expression was mediated by the activation of ERK1/2 and JNK1/2, and was unaffected by p38 MAPK and NFκB.

### Example 3

### Selectvie Regulation of Cytokine Expression by C-Myc

To investigate the role of c-Myc in regulating cytokine induction, PBMac were pre-incubated with control or c-Myc siRNA, and then treated with BCG or M. avium. The c-Myc siRNA significantly reduced the BCG-induced c-Myc protein levels after BCG addition, as compared to control siRNA treated cells (FIG. 3).

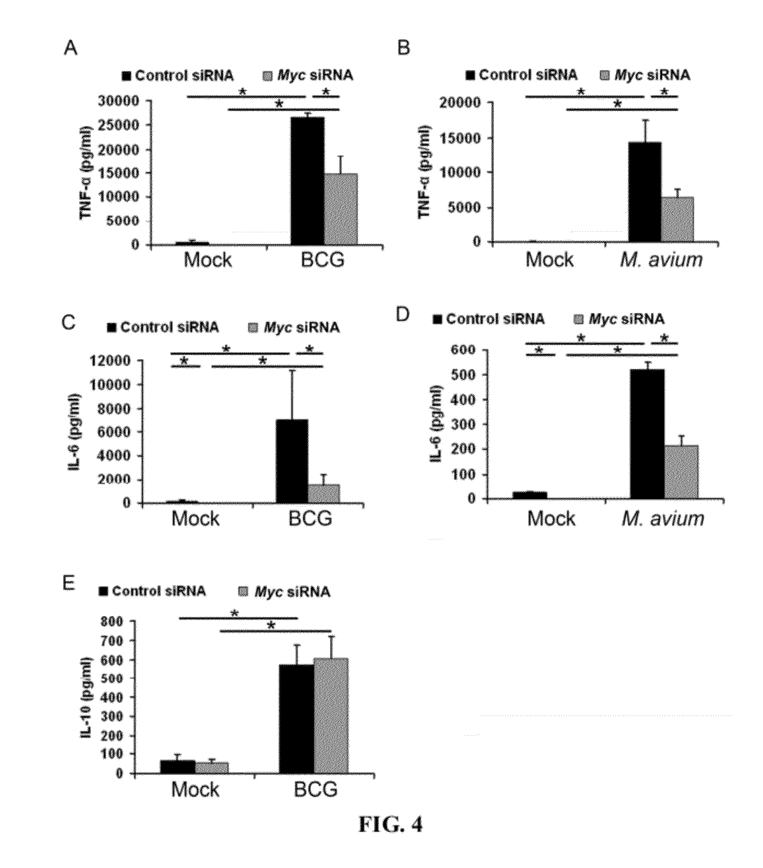

To investigate whether c-Myc modulates TNF-α expression, the culture media were harvested for measuring the cytokine levels by ELISA. The results showed that both BCG and M. avium induce the production of TNF-α protein in control or c-Myc siRNA-transfected cells, as compared to mock-treated cells (FIGS. 4A and 4B). However, the mycobacteria-induction of TNF-α protein expression was significantly decreased by c-Myc siRNA, as compared to the control siRNA-treated cells (FIGS. 4A and 4B).

Similarly, both BCG and M. avium induced IL-6 protein release in control or c-Myc siRNA-transfected cells, as compared to mock-treated cells (FIGS. 4C and 4D). However, c-Myc siRNA decreased the basal level of IL-6 protein and significantly reduced the mycobacteria-induced IL-6 release, as compared to control siRNA-treated cells (FIGS. 4C and 4D).

In contrast, while BCG stimulated IL-10 protein production in control and c-Myc siRNA-transfected cells, as compared to that of mock-treated cells, c-Myc siRNA affected neither the basal nor the mycobacteria-induced IL-10 protein level, as compared to the control siRNA-treated cells (FIG. 4E).

### Example 4

### Selective Regulation of Cytokine Transcription by C-Myc

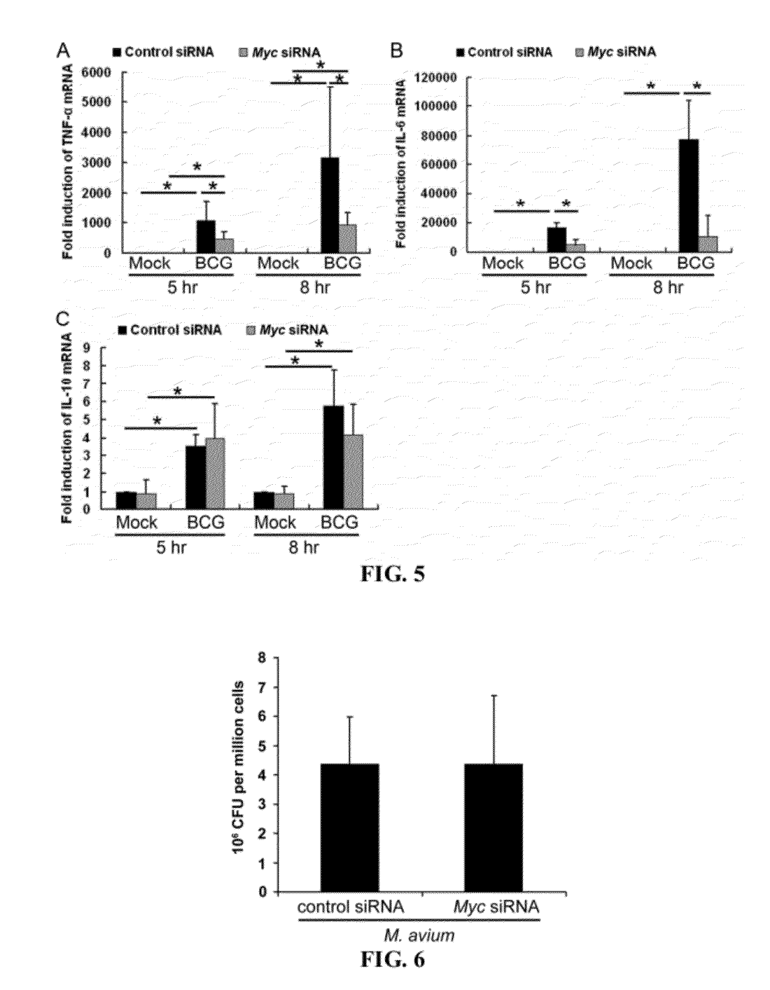

To determine whether c-Myc regulates the cytokine induction at the transcriptional level, PBMac transfected with control or c-Myc siRNA and treated with BCG as described above were assayed by quantitative RT-PCR. The results, as shown in FIGS. 5A-C, demonstrated that c-Myc was required for regulation of mycobacteria-induced TNF-α and IL-6 transcription, whereas c-Myc was not required for mycobacteria-induced IL-10 transcription.

Specifically, the results demonstrated that TNF-α mRNA levels increased at 5 and 8 hour after BCG addition in control or c-Myc siRNA-transfected cells, as compared to the mock cells (FIG. 5A). Treatment with c-Myc siRNA significantly reduced the mycobacteria-induced TNF-α mRNA transcription, as compared to the control siRNA treated-cells (FIG. 5A).

Similarly, IL-6 mRNA levels increased at 5 and 8 hour after BCG addition in control or c-Myc siRNA-transfected cells, as compared to the mock cells (FIG. 5B). Treatment with c-Myc siRNA significantly reduced the mycobacteria-induced IL-6 mRNA transcription, as compared to the control siRNA treated-cells (FIG. 5B).

In contrast, while IL-10 mRNA levels increased after BCG treatment, treatment with c-Myc siRNA did not affect mycobacteria-induced IL-10 mRNA expression (FIG. 5C).

### Example 5

### Effects of C-Myc on Survival of Mycobacterial During Acute Infection

To investigate whether c-Myc affects mycobacterial survival during acute infection, PBMac were transfected with control or c-Myc siRNA, followed by M. avium treatment for 24 hours. The intracellular number of M. avium was counted. The results showed comparable number of M. avium bacilli in control and c-Myc siRNA-transfected cells (FIG. 6), indicating that c-Myc did not directly affect the survival of mycobacterial during acute infection.

### Example 6

### Effects of C-Myc on PBMAC Viability During Acute Infection

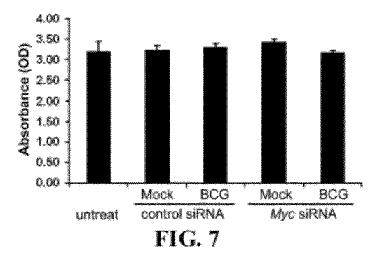

To examine the effects of c-Myc on PBMac viability during acute infection, PBMac were transfected with control or c-Myc siRNA, followed by incubation with mock or BCG for 24 hours. Cell viability was determined by the MTT assay. The viability of untreated, mock- and BCG-treated cells, with or without control and c-Myc siRNA transfection, were comparable (FIG. 7), indicating that c-Myc did not affect the viability of PBMac during acute infection.

### Example 7

### Regulation of Mycobacterial-Induced Cytokine Signaling Pathway

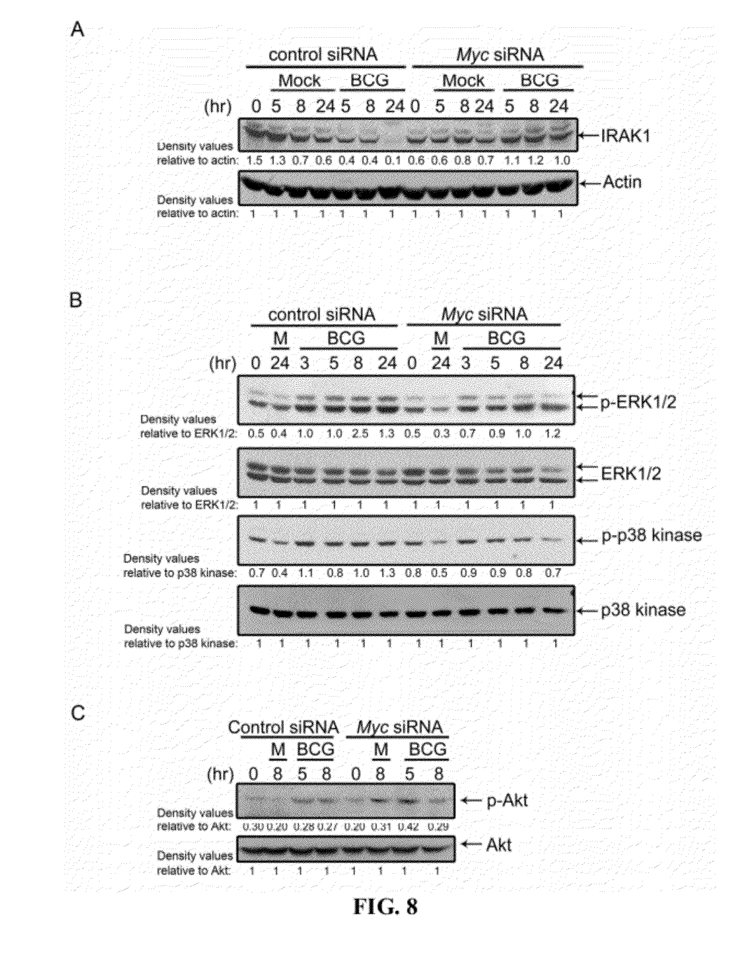

This Example examines the mechanisms through which c-Myc regulates mycobacteria-induced cytokine signaling pathways. Briefly, PBMac were transfected with control or c-Myc siRNA, followed by incubation with mock or BCG for 24 hours, and the levels of cytokine expression were analyzed by Western blots. The results, as shown in FIGS. 8A-C, indicate that c-Myc enhanced the activation of IRAK1 expression by mycobacteria. c-Myc also enhanced the phosphorylation of ERK1/2 and p38 kinases downstream of IRAK1 activation in the signaling pathway. c-Myc has little effect on the mycobacteria-induced phosphorylation of Akt.

Specifically, the results demonstrated that BCG induced the degradation of IRAK1 in a time-dependent manner starting from 5 hour and with the complete degradation at 24 hour (FIG. 8A). However, when the cells were transfected with c-Myc siRNA, the protein levels of IRAK1 substantially increased at 5, 8 and 24 hour, as compared to the control siRNA-transfected cells (FIG. 8A).

In addition, the role of c-Myc in regulating the downstream pathways of IRAK1 was studied. The results showed that the levels of phosphorylated ERK1/2 and p38 kinases increased from 3 to 24 hour after BCG treatment (FIG. 8B). The BCG-induced increase in the phosphorylation of ERK1/2 and p38 kinases was reduced by c-Myc siRNA (FIG. 8B).

In contrast, while the levels of phosphorylated Akt increased at 5 and 8 hour after BCG addition (FIG. 8C), such levels were only slightly increased by c-Myc siRNA (FIG. 8C). This indicates that c-Myc does not affect anti-inflammatory pathways.

### Example 8

### Localization of C-Myc in the Cytoplasm of Macrophages

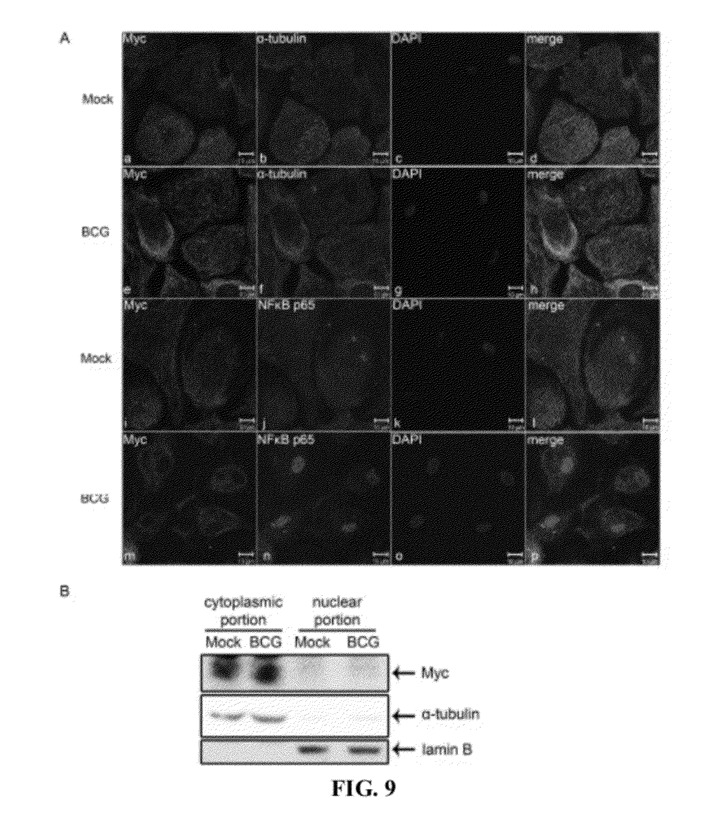

While previous studies have reported that c-Myc localizes in the nucleus and functions as a transcription factor to regulate various cellular processes (Dang and Lee, 1988; Meyer and Penn, 2008; Papoulas et al., 1992; Vriz et al., 1992), this Example investigates whether c-Myc localizes in the nucleus or cytoplasm of PBMac. The results, as shown by immunofluorescence confocal microscopy, revealed that c-Myc colocalized with α-tubulin, a cytoplasm marker, in the cytoplasm of mock and BCG-treated cells (FIG. 9A). Specifically, both constitutive and mycobacteria-induced c-Myc proteins reside in the cytoplasm not in the nucleus of PBMac.

As shown in FIG. 9, c-Myc also colocalized with NFκB p65 in the cytoplasm of mock-treated cells. Consistent with previous studies (Cheung et al., 2005; Darieva et al., 2004), the results showed that NFκB p65 was activated and translocated to the nucleus after BCG treatment. In contrast, c-Myc protein remained localized in the cytoplasm after BCG treatment.

The confocal images were further quantified by ImageJ software (National Institute of Health, USA) with Pearson-Spearman correlation colocalization plug-in as described (French et al., 2008; Pryor et al., 2008). Positive correlations of colocalization were found in merged images of panel a and b (rₚ=0.37, rₛ=0.52), e and f (rₚ=0.56, rₛ=0.58), and i and j (rₚ=0.3, rₛ=0.49) of FIG. 9A, while negative correlations of colocalization were found in merged image of panel m and n (rₚ=0.28, rₛ=0.33) of FIG. 9A. The results showed that c-Myc localized in the cytoplasm with α-tubulin with or without BCG treatment.

To further investigate the localization of c-Myc in the cytoplasm of PBMac, PBMac were treated with mock or BCG for 5 hours. Cytoplasmic and nuclear proteins were harvested for Western blot analyses. c-Myc proteins were found in the cytoplasm but not in the nucleus of mock or BCG-treated cells (FIG. 9B). Consistent with FIG. 1A, the c-Myc protein level in BCG-treated cells was higher than that in mock-treated cells (FIG. 9B). The results also confirmed the presence of a-tubulin, a cytoplasm-specific marker, in the cytoplasm, and the presence of lamin B, a specific nucleus marker, in the nucleus, but not vice versa (FIG. 9B). FIG. 9B showed the purity of the fractionated proteins.

### Example 9

### Interaction of C-Myc with IκBα

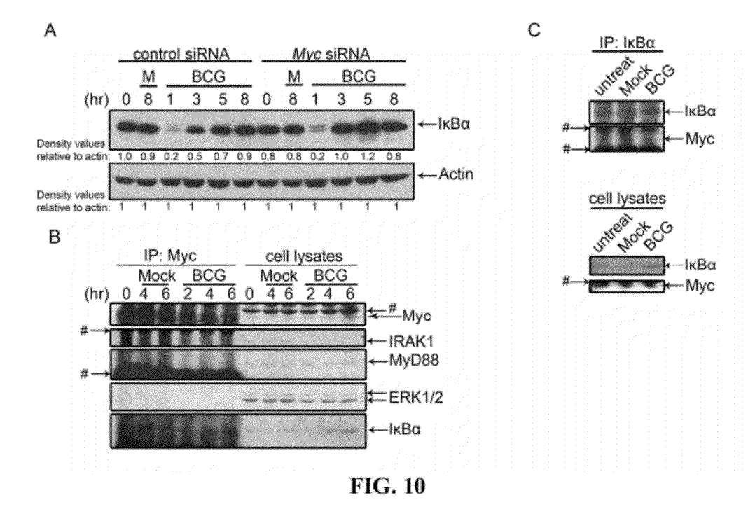

In addition to the MAPK pathway, c-Myc also modulates the IκBα pathway, another signaling pathway downstream of IRAK1. The results, as shown in FIG. 10, demonstrated that c-Myc binds to IκBα, but does not bind to IRAK1, MyD88 or ERK1/2.

Specifically, the results showed that IκBα protein levels decreased at 1 hour and then elevated at 3 to 8 hour after BCG treatment (FIG. 10A). The levels of IκBα were further upregulated by the c-Myc siRNA at 3 and 5 hour (FIG. 10A). Thus, c-Myc inhibited the mycobacteria-induced expression of IκBα protein.

To elucidate the interaction between c-Myc and various kinases, PBMacs were treated with or without BCG. Proteins extracted from cell lysates were immunoprecipitated by an anti-c-Myc monoclonal antibody and separated by Western blots. As a control, c-Myc was precipitated and detected by the anit-c-Myc antibody (FIG. 10B).

FIG. 10B showed that c-Myc did not bind to IRAK1. IRAK1 was not detected in anti-c-Myc antibody-precipitated proteins in mock- and BCG-treated cells, but was only found in non-precipitated proteins in untreated, mock- or BCG-treated cells (FIG. 10B).

Similarly, c-Myc interacts with neither MyD88 nor ERK1/2. MyD88 and ERK1/2 were only detected in the cell lysates with or without BCG treatment, but were not detected in the anti-c-Myc antibody immunoprecipitates (FIG. 10B).

In contrast, the results showed that c-Myc constitutively interacts with IκBα, regardless of BCG treatment. IκBα was detected in the anti-c-Myc antibody immunoprecipitates with or without BCG treatment (FIG. 10B). As a control, IκBα was also present in the cell lysates, with its level decreased at 2 hour after BCG addition and increased over the basal level starting from 4 hour after BCG treatment (FIG. 10B).

To confirm that c-Myc constitutively interacts with IKBa, PBMac were treated with or without BCG for protein extraction. IκBα proteins were immunoprecipitated by anti-IκBα antibodies. The results showed the presence of IκBα in the anti-IκBα-antibody-immunoprecipitated proteins (FIG. 10C, upper panel). c-Myc was also detected in the anti-IKBa-antibodies immunoprecipitated proteins isolated from cells with or without BCG treatment (FIG. 10C, upper panel). FIG. 10C, lower panel is presented as controls, showing that IκBα and c-Myc can be detected in cell lysates (FIG. 10C, lower panel).

### Example 10

### Interaction of C-Myc with NFκB

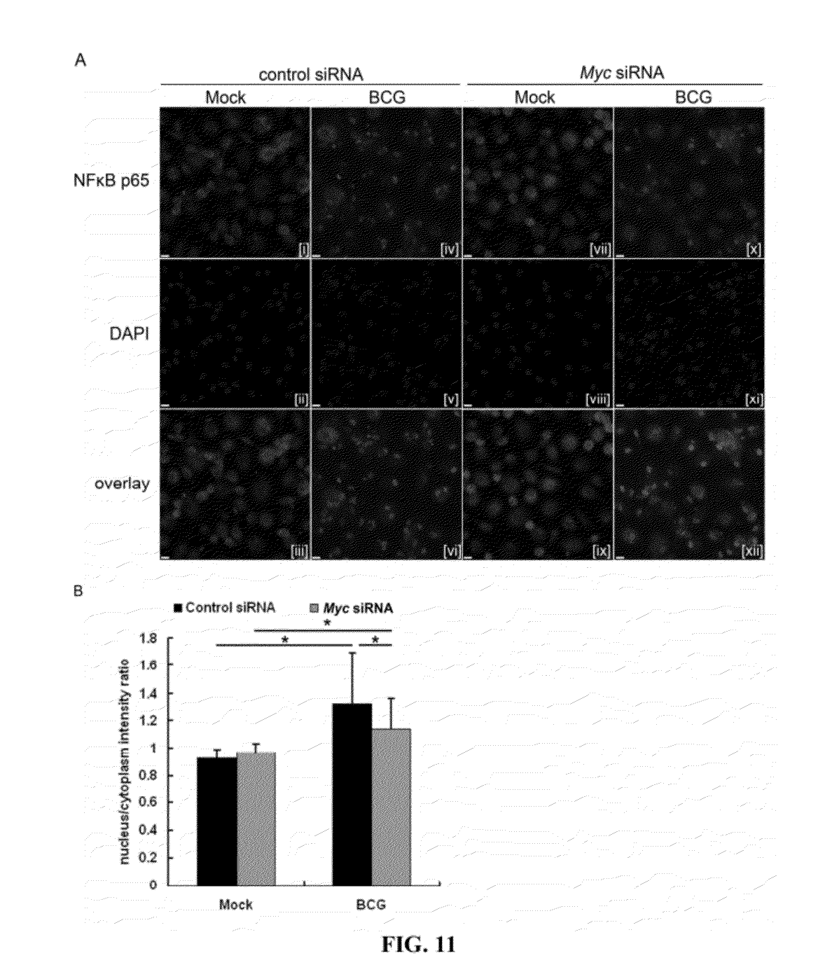

This Example investigates whether c-Myc regulates NFκB activation, another signaling pathway downstream of IRAK1. Briefly, the nuclear translocation of NFκB p65 was examined by immunocytochemistry with analysis using Cellomics ArrayScan HCS VTI Reader. As shown in FIG. 11A, BCG induced high levels of NFκB p65 nuclear localization, as compared to mock-treated cells (FIG. 11A). c-Myc siRNA significantly reduced the number of BCG-treated cells with NFκB p65 nuclear localization (FIG. 11A).

To further examine the induction of the nuclear localization of NFκB p65 by c-Myc, the results shown in FIG. 11A were quantified. FIG. 11B shows the increase of the nucleus/cytoplasm NFκB p65 intensity ratio induced by BCG, indicating that BCG triggered the nuclear localization of NFκB p65. In comparison, the BCG-induced increase of the nucleus/cytoplasm NFκB p65 intensity ratio was decreased by c-Myc siRNA. The results indicated that c-Myc augments the mycobacteria-induced NFκB nuclear translocation.

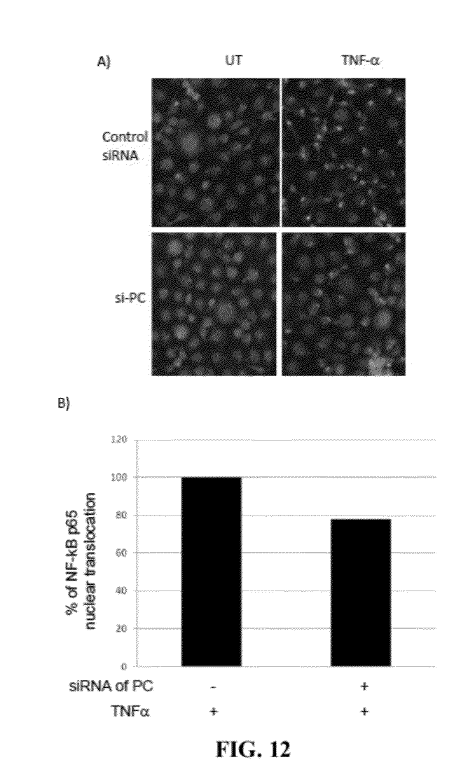

In addition, c-Myc plays a significant role in regulating the downstream cellular pathways controlled by TNF-α and NF-kB, in addition to its effect on cytokine regulation. Specifically, c-Myc promotes the TNF-α-induced NF-kB nuclear translocation (FIG. 12).

### Example 11

### Induction of TAT-Induced Cytokine Responses by C-Myc

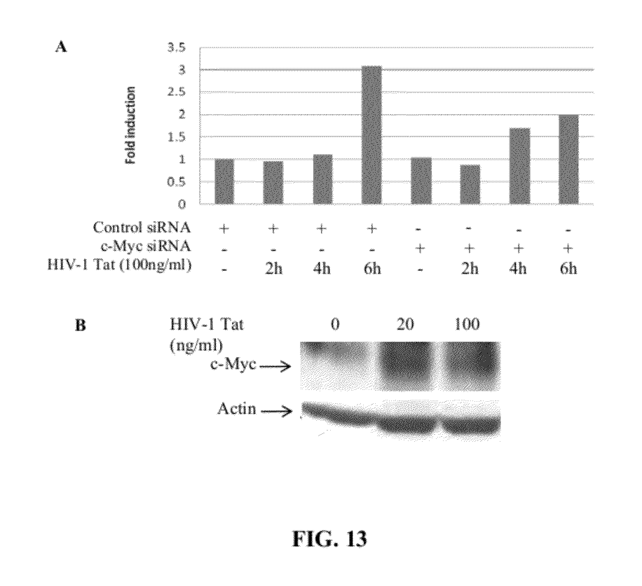

This Example shows that HIV-1 Tat induces c-Myc expression in primary human blood macrophages (FIG. 13). Briefly, PBMac were treated with or without Tat (100 g/ml) for indicated time points (2, 4 or 6 hours). Total RNA was extracted for analyzing c-Myc mRNA expression levels by quantitative RT-PCR. In addition, PBMac were treated with HIV-1 Tat (20 or 100 ng/ml) for 24 hours. Cellular proteins were extracted for Western blot analyses to determine protein levels of c-Myc and actin. The level of actin was measured as a control of the amount of proteins loaded.

### Example 12

### Mediation of TAT-induced Cytokine Responses by C-Myc

This Example shows that c-Myc mediates the induction of cytokines by HIV-1 Tat (FIG. 14). Briefly, PBMac were transfected with control or c-Myc siRNA for 48 hours, followed by treatment with HIV-1 Tat (100 or 200 ng/ml) for 24 hours. Supernatants were harvested for determining the levels of TNF-α (A), IL-6 (B) and IL-1β (C) by ELISA.

All references, including publications, patent applications and patents, cited herein are hereby incorporated by reference to the same extent as if each reference was individually and specifically indicated to be incorporated by reference and was set forth in its entirety herein.

The terms “a” and “an” and “the” and similar referents as used in the context of describing the invention are to be construed to cover both the singular and the plural, unless otherwise indicated herein or clearly contradicted by context.

Recitation of ranges of values herein are merely intended to serve as a shorthand method of referring individually to each separate value falling within the range, unless otherwise indicated herein, and each separate value is incorporated into the specification as if it were individually recited herein. Unless otherwise stated, all exact values provided herein are representative of corresponding approximate values (e.g., all exact exemplary values provided with respect to a particular factor or measurement can be considered to also provide a corresponding approximate measurement, modified by “about,” where appropriate).

The use of any and all examples, or exemplary language (e.g., “such as”) provided herein, is intended merely to better illuminate the invention and does not pose a limitation on the scope of the invention unless otherwise indicated. No language in the specification should be construed as indicating any element is essential to the practice of the invention unless as much is explicitly stated.

The description herein of any aspect or embodiment of the invention using terms such as “comprising”, “having”, “including” or “containing” with reference to an element or elements is intended to provide support for a similar aspect or embodiment of the invention that “consists of’, “consists essentially of', or “substantially comprises” that particular element or elements, unless otherwise stated or clearly contradicted by context (e.g., a composition described herein as comprising a particular element should be understood as also describing a composition consisting of that element, unless otherwise stated or clearly contradicted by context).

It should be understood that the examples and embodiments described herein are for illustrative purposes only and that various modifications or changes in light thereof will be suggested to persons skilled in the art and are to be included within the spirit and purview of this application.

### REFERENCES

Alter-Koltunoff, M., Goren, S., Nousbeck, J., Feng, C. G., Sher, A., Ozato, K., Azriel, A., and Levi, B. Z. (2008). Innate immunity to intraphagosomal pathogens is mediated by interferon regulatory factor 8 (IRF-8) that stimulates the expression of macrophage-specific Nramp1 through antagonizing repression by c-Myc. J Biol Chem 283, 2724-2733.
Amati, B. (2004). Myc degradation: dancing with ubiquitin ligases. Proc Natl Acad Sci USA 101, 8843-8844.

Bafica, A., Scanga, C. A., Feng, C. G., Leifer, C., Cheever, A., and Sher, A. (2005). TLR9 regulates Thl responses and cooperates with TLR2 in mediating optimal resistance to Mycobacterium tuberculosis. J Exp Med 202, 1715-1724.
Banerjee, A., Gugasyan, R., McMahon, M., and Gerondakis, S. (2006). Diverse Toll-like receptors utilize Tpl2 to activate extracellular signal-regulated kinase (ERK) in hemopoietic cells. Proc Natl Acad Sci USA 103, 3274-3279.
Bettelli, E., Korn, T., Oukka, M., and Kuchroo, V. K. (2008). Induction and effector functions of T(H)17 cells. Nature 453, 1051-1057.

Blumenthal, A., Lauber, J., Hoffmann, R., Ernst, M., Keller, C., Buer, J., Ehlers, S., and Reiling, N. (2005). Common and unique gene expression signatures of human macrophages in response to four strains of Mycobacterium avium that differ in their growth and persistence characteristics. Infect Immun 73, 3330-3341.

Bowen, H., Biggs, T. E., Phillips, E., Baker, S. T., Perry, V. H., Mann, D. A., and Barton, C. H. (2002). c-Myc represses and Miz-1 activates the murine natural resistance-associated protein 1 promoter. J Biol Chem 277, 34997-35006.
Brooks, T. A., and Hurley, L. H. (2009). The role of supercoiling in transcriptional control of MYC and its importance in molecular therapeutics. Nat Rev Cancer 9, 849-861.
Chan, L. L., Cheung, B. K., Li, J. C., and Lau, A. S. (2010). A role for STAT3 and cathepsin S in IL-10 down-regulation of IFN-{gamma}-induced MHC class II molecule on primary human blood macrophages. J Leukoc Biol.
Chan, M. M., Cheung, B. K., Li, J. C., Chan, L. L., and Lau, A. S. (2009). A role for glycogen synthase kinase-3 in antagonizing mycobacterial immune evasion by negatively regulating IL-10 induction. J Leukoc Biol 86, 283-291.
Cheng, S. M., Li, J. C., Lin, S. S., Lee, D. C., Liu, L., Chen, Z., and Lau, A. S. (2009). HIV-1 transactivator protein induction of suppressor of cytokine signaling-2 contributes to dysregulation of IFN {gamma} signaling. Blood 113, 5192-5201.
Cheng, S. M., Xing, B., Li, J. C., Cheung, B. K., and Lau, A. S. (2007). Interferon-gamma regulation of TNFalpha-induced matrix metalloproteinase 3 expression and migration of human glioma T98G cells. Int J Cancer 121, 1190-1196.
Cheng, Y., Chaturvedi, R., Asim, M., Bussiere, F. I., Scholz, A., Xu, H., Casero, R. A., Jr., and Wilson, K. T. (2005). Helicobacter pylori-induced macrophage apoptosis requires activation of ornithine decarboxylase by c-Myc. J Biol Chem 280, 22492-22496.
Cheung, B. K., Lee, D. C., Li, J. C., Lau, Y. L., and Lau, A. S. (2005). A role for double-stranded RNA-activated protein kinase PKR in Mycobacterium-induced cytokine expression. J Immunol 175, 7218-7225.
Cheung, B. K., Yim, H. C., Lee, N. C., and Lau, A. S. (2009). A novel anti-mycobacterial function of mitogen-activated protein kinase phosphatase-1. BMC Immunol 10, 64.
Cheng, S. M., Li, J. C., Lin, S. S., Lee, D. C., Liu, L., Chen, Z., and Lau, A. S. (2009). HIV-1 transactivator protein induction of suppressor of cytokine signaling-2 contributes to dysregulation of IFN {gamma}signaling. Blood 113, 5192-5201.

Cho, J., Melnick, M., Solidakis, G. P., and Tsichlis, P. N. (2005). Tp12 (tumor progression locus 2) phosphorylation at Thr290 is induced by lipopolysaccharide via an Ikappa-B Kinase-beta-dependent pathway and is required for Tp12 activation by external signals. J Biol Chem 280, 20442-20448.
Cho, J., and Tsichlis, P. N. (2005). Phosphorylation at Thr-290 regulates Tp12 binding to NF-kappaB1/p105 and Tp12 activation and degradation by lipopolysaccharide. Proc Natl Acad Sci USA 102, 2350-2355.
Cooper, A. M., and Khader, S. A. (2008). The role of cytokines in the initiation, expansion, and control of cellular immunity to tuberculosis. Immunol Rev 226, 191-204.

Craig, R. W., Buchan, H. L., Civin, C. I., and Kastan, M. B. (1993). Altered cytoplasmic/nuclear distribution of the c-myc protein in differentiating ML-1 human myeloid leukemia cells. Cell Growth Differ 4, 349-357.
Dang, C. V., and Lee, W. M. (1988). Identification of the human c-myc protein nuclear translocation signal. Mol Cell Biol 8, 4048-4054.
Darieva, Z., Lasunskaia, E. B., Campos, M. N., Kipnis, T. L., and Da Silva, W. D. (2004). Activation of phosphatidylinositol 3-kinase and c-Jun-N-terminal kinase cascades enhances NF-kappaB-dependent gene transcription in BCG-stimulated macrophages through promotion of p65/p300 binding. J Leukoc Biol 75, 689-697.
Davis, A. C., Wims, M., Spotts, G. D., Hann, S. R., and Bradley, A. (1993). A null c-myc mutation causes lethality before 10.5 days of gestation in homozygotes and reduced fertility in heterozygous female mice. Genes Dev 7, 671-682.
de Alboran, I. M., O'Hagan, R. C., Gartner, F., Malynn, B., Davidson, L., Rickert, R., Rajewsky, K., DePinho, R. A., and Alt, F. W. (2001). Analysis of C-MYC function in normal cells via conditional gene-targeted mutation. Immunity 14, 45-55.

Ehlers, S. (2003). Role of tumour necrosis factor (TNF) in host defence against tuberculosis: implications for immunotherapies targeting TNF. Ann Rheum Dis 62 Suppl 2, ii37-42.
Fernandez, P. C., Frank, S. R., Wang, L., Schroeder, M., Liu, S., Greene, J., Cocito, A., and Amati, B. (2003). Genomic targets of the human c-Myc protein. Genes Dev 17, 1115-1129.
Ferwerda, G., Girardin, S. E., Kullberg, B. J., Le Bourhis, L., de Jong, D. J., Langenberg, D. M., van Crevel, R., Adema, G. J., Ottenhoff, T. H., Van der Meer, J. W., and Netea, M. G. (2005). NOD2 and toll-like receptors are nonredundant recognition systems of Mycobacterium tuberculosis. PLoS Pathog 1, 279-285.
Flynn, J. L., Goldstein, M. M., Chan, J., Triebold, K. J., Pfeffer, K., Lowenstein, C. J., Schreiber, R., Mak, T. W., and Bloom, B. R. (1995). Tumor necrosis factor-alpha is required in the protective immune response against Mycobacterium tuberculosis in mice. Immunity 2, 561-572.
French, A. P., Mills, S., Swamp, R., Bennett, M. J., and Pridmore, T. P. (2008). Colocalization of fluorescent markers in confocal microscope images of plant cells. Nat Protoc 3, 619-628.
Fukumori T, Akari H, Iida S, Hata S, Kagawa S, Aida Y, Koyama A H, Adachi A. The HIV-1 Vpr displays strong anti-apoptotic activity. FEBS Lett. 1998. 432:17-20. 30. Strebel K. Virus-host interactions: role of H1V proteins Vif, Tat, and Rev. AIDS 2003. 17 (Suppl. 4): S25-34.
Fukunaga, R., and Hunter, T. (1997). MNK1, a new MAP kinase-activated protein kinase, isolated by a novel expression screening method for identifying protein kinase substrates. EMBO J 16, 1921-1933.
Gao, P., Tchernyshyov, I., Chang, T. C., Lee, Y. S., Kita, K., Ochi, T., Zeller, K. I., De Marzo, A. M., Van Eyk, J. E., Mendell, J. T., and Dang, C. V. (2009). c-Myc suppression of miR-23a/b enhances mitochondrial glutaminase expression and glutamine metabolism. Nature 458, 762-765.
Garrison, B. S., and Rossi, D. J. (2010). Controlling stem cell fate one substrate at a time. Nat Immunol 11, 193-194.
Ghosh, S., and Hayden, M. S. (2008). New regulators of NF-kappaB in inflammation. Nat Rev Immunol 8, 837-848.

Greenwell-Wild, T., Vazquez, N., Sim, D., Schito, M., Chatterjee, D., Orenstein, J. M., and Wahl, S. M. (2002). Mycobacterium avium infection and modulation of human macrophage gene expression. J Immunol 169, 6286-6297.

Heldwein, K. A., Liang, M. D., Andresen, T. K., Thomas, K. E., Marty, A. M., Cuesta, N., Vogel, S. N., and Fenton, M. J. (2003). TLR2 and TLR4 serve distinct roles in the host immune response against Mycobacterium bovis BCG. J Leukoc Biol 74, 277-286.

Hu, X., Paik, P. K., Chen, J., Yarilina, A., Kockeritz, L., Lu, T. T., Woodgett, J. R., and Ivashkiv, L. B. (2006). IFN-gamma suppresses IL-10 production and synergizes with TLR2 by regulating GSK3 and CREB/AP-1 proteins. Immunity 24, 563-574.
Huang, Q., Yang, J., Lin, Y., Walker, C., Cheng, J., Liu, Z. G., and Su, B. (2004). Differential regulation of interleukin 1 receptor and Toll-like receptor signaling by MEKK3. Nat Immunol 5, 98-103.
Hurlin P J, Huang J. (2006). The MAX-interacting transcription factor network. Semin Cancer Biol. 2006 August; 16(4):265-74.

Jacobs, M., Marino, M. W., Brown, N., Abel, B., Bekker, L. G., Quesniaux, V. J., Fick, L., and Ryffel, B. (2000). Correction of defective host response to Mycobacterium bovis BCG infection in TNF-deficient mice by bone marrow transplantation. Lab Invest 80, 901-914.
Jang, S., Uematsu, S., Akira, S., and Salgame, P. (2004). IL-6 and IL-10 induction from dendritic cells in response to Mycobacterium tuberculosis is predominantly dependent on TLR2-mediated recognition. J Immunol 173, 3392-3397.
Kang, Y. J., Kim, S. O., Shimada, S., Otsuka, M., Seit-Nebi, A., Kwon, B. S., Watts, T. H., and Han, J. (2007). Cell surface 4-1BBL mediates sequential signaling pathways ‘downstream’ of TLR and is required for sustained TNF production in macrophages. Nat Immunol 8, 601-609.
Karakousis, P. C., Moore, R. D., and Chaisson, R. E. (2004). Mycobacterium avium complex in patients with HIV infection in the era of highly active antiretroviral therapy. Lancet Infect Dis 4, 557-565.
Kasper D L, Braunwald E, Fauci A S, Hauser S, Longo D, Jameson J L, (Ed.) Harrison's Principles of Internal Medicine 16th edition 2005. pp1071-1139.
Kaufmann, S. H., and McMichael, A. J. (2005). Annulling a dangerous liaison: vaccination strategies against AIDS and tuberculosis. Nat Med 11, S33-44.
Laurenti, E., Wilson, A., and Trumpp, A. (2009). Myc's other life: stem cells and beyond. Curr Opin Cell Biol 21, 844-854.
Lee, D. C., Cheung, C. Y., Law, A. H., Mok, C. K., Peiris, M., and Lau, A. S. (2005). p38 mitogen-activated protein kinase-dependent hyperinduction of tumor necrosis factor alpha expression in response to avian influenza virus H5N1. J Virol 79, 10147-10154.
Leemans, J. C., Thepen, T., Weijer, S., Florquin, S., van Rooijen, N., van de Winkel, J. G., and van der Poll, T. (2005). Macrophages play a dual role during pulmonary tuberculosis in mice. J Infect Dis 191, 65-74.
Li, J. C., and Lau, A. S. (2007). A role for mitogen-activated protein kinase and Ets-1 in the induction of interleukin-10 transcription by human immunodeficiency virus-1 Tat. Immunology 121, 337-348.
Li J C, Lee D C, Cheung B K, and Lau A S. Mechanisms for HIV Tat upregulation of IL-10 and other cytokine expression: kinase signaling and PKR-mediated immune response. FEBS Lett. 2005. 579:3055-3062.
Li J C, Yim H C, Lau A S. (2010). Role of HIV-1 Tat in AIDS pathogenesis: its effects on cytokine dysregulation and contributions to the pathogenesis of opportunistic infection. AIDS. 24(11):1609-23.
Li, Z., Van Calcar, S., Qu, C., Cavenee, W. K., Zhang, M. Q., and Ren, B. (2003). A global transcriptional regulatory role for c-Myc in Burkitt's lymphoma cells. Proc Natl Acad Sci USA 100, 8164-8169.
Litvak, V., Ramsey, S. A., Rust, A. G., Zak, D. E., Kennedy, K. A., Lampano, A. E., Nykter, M., Shmulevich, I., and Aderem, A. (2009). Function of C/EBPdelta in a regulatory circuit that discriminates between transient and persistent TLR4-induced signals. Nat Immunol 10, 437-443.
Maranon, F., Chen, X., Lee, A. H., and Glimcher, L. H. (2010). TLR activation of the transcription factor XBP1 regulates innate immune responses in macrophages. Nat Immunol 11, 411-418.
McMichael A J, and Rowland-Jones S L. Cellular immune responses to HIV. Nature 2001. 410:980-987.
McMahon, S. B., Van Buskirk, H. A., Dugan, K. A., Copeland, T. D., and Cole, M. D. (1998). The novel ATM-related protein TRRAP is an essential cofactor for the c-Myc and E2F oncoproteins. Cell 94, 363-374.
McMahon, S. B., Wood, M. A., and Cole, M. D. (2000). The essential cofactor TRRAP recruits the histone acetyltransferase hGCN5 to c-Myc. Mol Cell Biol 20, 556-562.
Mege J L, Meghari S, Honstettre A, Capo C, and Raoult D. The two faces of interleukin 10 in human infectious diseases. Lancet Infect Dis. 2006. 6:557-569.
Meyer, N., and Penn, L. Z. (2008). Reflecting on 25 years with MYC. Nat Rev Cancer 8, 976-990.
Moynagh, P. N. (2009). The Pellino family: IRAK E3 ligases with emerging roles in innate immune signalling. Trends Immunol 30, 33-42.
Nakagawa, M., Koyanagi, M., Tanabe, K., Takahashi, K., Ichisaka, T., Aoi, T., Okita, K., Mochiduki, Y., Takizawa, N., and Yamanaka, S. (2008). Generation of induced pluripotent stem cells without Myc from mouse and human fibroblasts. Nat Biotechnol 26, 101-106.
Niklinski, J., Claassen, G., Meyers, C., Gregory, M. A., Allegra, C. J., Kaye, F. J., Hann, S. R., and Zajac-Kaye, M. (2000). Disruption of Myc-tubulin interaction by hyperphosphorylation of c-Myc during mitosis or by constitutive hyperphosphorylation of mutant c-Myc in Burkitt's lymphoma. Mol Cell Biol 20, 5276-5284.
Noursadeghi M, Katz D R, and Miller R F. HIV-1 infection of mononuclear phagocytic cells: the case for bacterial innate immune deficiency in AIDS. Lancet Infect Dis. 2006. 6:794-804.
O'Neill, L. A., and Bowie, A. G. (2007). The family of five: TIR-domain-containing adaptors in Toll-like receptor signalling. Nat Rev Immunol 7, 353-364.
Okita, K., Ichisaka, T., and Yamanaka, S. (2007). Generation of germline-competent induced pluripotent stem cells. Nature 448, 313-317.
Papoulas, O., Williams, N. G., and Kingston, R. E. (1992). DNA binding activities of c-Myc purified from eukaryotic cells. J Biol Chem 267, 10470-10480.
Peukert, K., Staller, P., Schneider, A., Carmichael, G., Hanel, F., and Eilers, M. (1997). An alternative pathway for gene regulation by Myc. EMBO J 16, 5672-5686.
Pryor, P. R., Jackson, L., Gray, S. R., Edeling, M. A., Thompson, A., Sanderson, C. M., Evans, P. R., Owen, D. J., and Luzio, J. P. (2008). Molecular basis for the sorting of the SNARE VAMP7 into endocytic clathrin-coated vesicles by the ArfGAP Hrb. Cell 134, 817-827.
Reavie, L., Della Gatta, G., Crusio, K., Aranda-Orgilles, B., Buckley, S. M., Thompson, B., Lee, E., Gao, J., Bredemeyer, A. L., Helmink, B. A., et al. (2010). Regulation of hematopoietic stem cell differentiation by a single ubiquitin ligase-substrate complex. Nat Immunol 11, 207-215.
Russell, D. G. (2007). Who puts the tubercle in tuberculosis? Nat Rev Microbiol 5, 39-47.
Seong, J., Piao, H. H., Ryu, P. Y., Kim, Y. U., Choy, H. E., and Hong, Y. (2009). Expression of c-Myc is related to host cell death following Salmonella typhimurium infection in macrophage. J Microbiol 47, 214-219.
Song, C. H., Lee, J. S., Lee, S. H., Lim, K., Kim, H. J., Park, J. K., Paik, T. H., and Jo, E. K. (2003). Role of mitogen-activated protein kinase pathways in the production of tumor necrosis factor-alpha, interleukin-10, and monocyte chemotactic protein-1 by Mycobacterium tuberculosis H37Rv-infected human monocytes. J Clin Immunol 23, 194-201.
Soucek, L., Whitfield, J., Martins, C. P., Finch, A. J., Murphy, D. J., Sodir, N. M., Karnezis, A. N., Swigart, L. B., Nasi, S., and Evan, G. I. (2008). Modelling Myc inhibition as a cancer therapy. Nature 455, 679-683.
Teitelbaum, R., Schubert, W., Gunther, L., Kress, Y., Macaluso, F., Pollard, J. W., McMurray, D. N., and Bloom, B. R. (1999). The M cell as a portal of entry to the lung for the bacterial pathogen Mycobacterium tuberculosis. Immunity 10, 641-650.
Thompson, M. R., Xu, D., and Williams, B. R. (2009). ATF3 transcription factor and its emerging roles in immunity and cancer. J Mol Med 87, 1053-1060.
Trumpp, A., Refaeli, Y., Oskarsson, T., Gasser, S., Murphy, M., Martin, G. R., and Bishop, J. M. (2001). c-Myc regulates mammalian body size by controlling cell number but not cell size. Nature 414, 768-773.
Underhill, D. M., Ozinsky, A., Smith, K. D., and Aderem, A. (1999). Toll-like receptor-2 mediates mycobacteria-induced proinflammatory signaling in macrophages. Proc Natl Acad Sci USA 96, 14459-14463.
Vennstrom, B., Sheiness, D., Zabielski, J., and Bishop, J. M. (1982). Isolation and characterization of c-myc, a cellular homolog of the oncogene (v-myc) of avian myelocytomatosis virus strain 29. J Virol 42, 773-779.
Vriz, S., Lemaitre, J. M., Leibovici, M., Thierry, N., and Mechali, M. (1992). Comparative analysis of the intracellular localization of c-Myc, c-Fos, and replicative proteins during cell cycle progression. Mol Cell Biol 12, 3548-3555.
Wernig, M., Meissner, A., Foreman, R., Brambrink, T., Ku, M., Hochedlinger, K., Bernstein, B. E., and Jaenisch, R. (2007). In vitro reprogramming of fibroblasts into a pluripotent ES-cell-like state. Nature 448, 318-324.

Wilson, A., Murphy, M. J., Oskarsson, T., Kaloulis, K., Bettess, M. D., Oser, G M., Pasche, A. C., Knabenhans, C., Macdonald, H. R., and Trumpp, A. (2004). c-Myc controls the balance between hematopoietic stem cell self-renewal and differentiation. Genes Dev 18, 2747-2763.
World Health Organization. (2009). Global tuberculosis control : epidemiology, strategy, financing : WHO report 2009 (Geneva: World Health Organization).
Yadav, M., and Schorey, J. S. (2006). The beta-glucan receptor dectin-1 functions together with TLR2 to mediate macrophage activation by mycobacteria. Blood 108, 3168-3175.
Yim, H. C., Li, J. C., Lau, J. S., and Lau, A. S. (2009). HIV-1 Tat dysregulation of lipopolysaccharide-induced cytokine responses: microbial interactions in HIV infection. AIDS 23, 1473-1484.
Zeller, K. I., Jegga, A. G., Aronow, B. J., O'Donnell, K. A., and Dang, C. V. (2003). An integrated database of genes responsive to the Myc oncogenic transcription factor: identification of direct genomic targets. Genome Biol 4, R69.
Zhu J, Blenis J, J Y Yuan (2008). Myc in Oncogenesis and as a target for cancer therapies. Proc Natl Acad Sci. 2008; 105: 6584-6589.

What is claimed is:

1. A method for treating inflammation and/or auto-immune diseases, wherein the method comprises reducing cytoplasmic c-Myc protein level and/or inhibiting c-Myc activity in the cytoplasm in a subject in need of such treatment.

2. The method according to claim 1, comprising administering to the subject an effective amount of a therapeutic agent selected from the group consisting of:
a) S2T1-6OTD, Quarfloxin (CX-3543), benzoylanthranilic acid, Cationic Porphyrin TMPyP4;
b) a c-Myc-specific siRNA;
c) an antibody, aptamer, or binding partner that binds specifically to c-Myc; and
d) a c-Myc antagonist selected from MAD1, MAX, MNT, MXD1-4 or MGA.

3. The method according to claim 1, wherein c-Myc protein level in the cytoplasm is reduced.

4. The method according to claim 1, wherein binding of c-Myc to IκBα is inhibited.

5. The method according to claim 1, wherein the method does not comprise administering an agent that inhibits or reduces c-Myc expression; and/or an agent that inhibits binding of c-Myc to Max to form c-Myc/Max heterodimeric transcription factor.

6. The method according to claim 1, wherein TNF-α level in the subject is reduced.

7. The method according to claim 1, wherein IL6 level in the subject is reduced.

8. The method according to claim 1, wherein ERK1/2 phosphorylation and/or p38 phosphorylation in the subject is reduced.

9. The method according to claim 1, wherein NFκB p65 nuclear translocation in the subject is reduced.

10. The method according to claim 1, used to treat infection, inflammation, allergenic reaction, neoangiogenesis, cardiovascular disease, and/or an autoimmune disease.

11. The method according to claim 10, used to treat inflammation associated with viral, bacterial, fungal, or protozoan infection.

12. The method according to claim 11, used to treat inflammation associated with mycobateria infection.

13. The method according to claim 10, used to treat HIV infection, opportunistic infection concurrent with HIV infection, or AIDS-associated disorders.

14. The method according to claim 13, used to concurrent infection by mycobacteria, Candida, Staphylococcus aureus, Salmonella, Escherichii coli, Listeria monocytogenes, Listeria amazonensis, HSV, HPV, KSHV, or hepatitis virus.

15. The method according to claim 5, used to treat cancer.

16. A method for enhancing pro-inflammatory immune activity, comprising administering, to a subject in need of such treatment, an effective amount of a c-Myc protein and/or a nucleic acid encoding a c-Myc protein.

17. A method for screening for therapeutic agents for treating inflammation or autoimmune diseases, comprising:
a) contacting a candidate agent with cells expressing c-Myc;
b) determining a level of c-Myc in the cytoplasm of the cells; and
c) selecting the candidate agent if said agent reduces the level of c-Myc in the cytoplasm.

18. The method according to claim 17, further comprising, after step a), the steps of:
determining a level of c-Myc expression; and
selecting the candidate agent if said agent does not reduce c-Myc expression.

19. The method according to claim 17, further comprising:
determining a level of a pro-inflammatory mediator in the cells; and
selecting the candidate agent if said agent reduces the level of the pro-inflammatory mediator;
wherein the pro-inflammatory mediator is TNF-α, IL-6, IL-2, and/or TGF-β.

20. The method according to claim 17, further comprising:
determining a level of IRAK1 in the cells; and
selecting the candidate agent if said agent reduces the level of IRAK1.

21. The method according to claim 17, further comprising:
determining a level of phosphorylated ERK1/2 and/or phosphorylated p38 in the cells; and
selecting the candidate agent if said agent reduces the level of phosphorylated ERK1/2 and/or phosphorylated p38.

22. The method according to claim 17, further comprising:
determining a level of IκBα in the cells; and
selecting the candidate agent if said agent reduces the level of IκBα.

23. The method according to claim 17, further comprising:
determining nucleus/cytoplasm NFκB p65 intensity ratio in the cells;
and selecting the candidate agent if said agent reduces the nucleus/cytoplasm NFκB p65 intensity ratio.

24. A method for screening for therapeutic agents for treating inflammation or autoimmune diseases, comprising:
a) contacting a candidate agent with cells expressing c-Myc, wherein the c-Myc is present in the cytoplasm;
b) determining binding of c-Myc to IxBa; and
c) selecting the candidate agent if said agent inhibits or reduces binding of c-Myc to IκBα.

25. A method for screening for therapeutic agents for enhancing innate pro-inflammatory responses, comprising:
a) contacting a candidate agent with cells expressing c-Myc;
b) determining a level of c-Myc in the cytoplasm of the cells; and
c) selecting the candidate agent if said agent increases the level of c-Myc in the cytoplasm.

26. The method according to claim 25, further comprising:
determining a level of a pro-inflammatory mediator in the cells; and
selecting the candidate agent if said agent increases the level of the pro-inflammatory mediator;
wherein the pro-inflammatory mediator is TNF-α, IL-6, IL-2, and/or TGF-β.

27. The method according to claim 25, further comprising:
determining a level of phosphorylated ERK1/2 and/or phosphorylated p38 in the cells; and
selecting the candidate agent if said agent increases the level of phosphorylated ERK1/2 and/or phosphorylated p38.


SEQUENCE LISTING (15 sequences)

SEQ ID NO: 1 - RNA, 19 nt, Artificial Sequence
  SiRNA
acggaacucuugugcguaa

SEQ ID NO: 2 - RNA, 19 nt, Artificial Sequence
  SiRNA
gaacacacaacgucuugga

SEQ ID NO: 3 - RNA, 19 nt, Artificial Sequence
  SiRNA
aacguuagcuucaccaaca

SEQ ID NO: 4 - RNA, 19 nt, Artificial Sequence
  SiRNA
cgauguuguuucuguggaa

SEQ ID NO: 5 - DNA, 21 bp, Artificial Sequence
  SiRNA
aaccuuggcugagucuugatt

SEQ ID NO: 6 - DNA, 21 bp, Artificial Sequence
  SiRNA
ucaagacucagccaagguutt

SEQ ID NO: 7 - DNA, 20 bp, Artificial Sequence
  SiRNA
acgttgaggggcatcgtcgc

SEQ ID NO: 8 - DNA, 30 bp, Artificial Sequence
  SiRNA
caggacccgcuucucugaaaggcucuccuu

SEQ ID NO: 9 - DNA, 25 bp, Artificial Sequence
  SiRNA
gcgacgaugccccucaacguuagcu

SEQ ID NO: 10 - DNA, 26 bp, Artificial Sequence
  SiRNA
aacuaugaccucgacuacgacucggu

SEQ ID NO: 11 - DNA, 28 bp, Artificial Sequence
  SiRNA
acugcgacgaggaggagaacuucuacca

SEQ ID NO: 12 - DNA, 15 bp, Artificial Sequence
  SiRNA
aacgttgaggggcat

SEQ ID NO: 13 - DNA, 20 bp, Artificial Sequence
  SiRNA
gggagagtcgcgtccttgct

SEQ ID NO: 14 - DNA, 20 bp, Artificial Sequence
  SiRNA
tcatggagcaccaggggctc

SEQ ID NO: 15 - protein, 439 aa, Artificial Sequence
  human c-Myc protein
MPLNVSFTNRNYDLDYDSVQPYFYCDEEENFYQQQQQSELQPPAPSEDIWKKFELLPTPPLSPSRRSGLCSPSYVAVTPFSLRGDNDGGGGSFSTADQLEMVTELLGGDMVNQSFICDPDDETFIKNIIIQDCMWSGFSAAAKLVSEKLASYQAARKDSGSPNPARGHSVCSTSSLYLQDLSAAASECIDPSVVFPYPLNDSSSPKSCASQDSSAFSPSSDSLLSSTESSPQGSPEPLVLHEETPPTTSSDSEEEQEDEEEIDVVSVEKRQAPGKRSESGSPSAGGHSKPPHSPLVLKRCHVSTHQHNYAAPPSTRKDYPAAKRVKLDSVRVLRQISNNRKCTSPRSSDTEENVKRRTHNVLERQRRNELKRSFFALRDQIPELENNEKAPKVVILKKATAYILSVQAEEQKLISEEDLLRKRREQLKHKLEQLRNSCA